(19) United States
(12) Patent Application Publication
(10) Pub. No.: US 2009/0325858 A1
(43) Pub. Date: Dec. 31, 2009

(54) Reduction of Zinc-Induced Neurotoxic Injury By Blockade of Nitric Oxide Synthesis

(75) Inventor: Christopher J. Frederickson, Galveston, TX (US)

Correspondence Address: Don D. Cha, 547 Buena Vista Road, Golden, CO, 80401, US

(73) Assignee: ANDRO DIAGNOSTICS, INC., Galveston, TX (US)

(21) Appl. No.: 12/478,751

(22) Filed: Jun. 4, 2009

Related U.S. Application Data
(63) Continuation of application No. 10/805,882, filed on Mar. 22, 2004, now abandoned.
(60) Provisional application No. 60/456,392, filed on Mar. 21, 2003.

(51) Int. Cl. A61K 38/02

(52) U.S. Cl. 514/2

(57) ABSTRACT

The present invention provides methods of inhibiting release of zinc from neurons and of preventing zinc-mediated brain injury. Also provided are methods of improving cerebral blood flow while preventing zinc-mediated brain injury. Inhibitors of or activators of nitric oxide synthases modulate nitric oxide synthesis to reduce nitric oxide-induced release of neurotoxic amounts of zinc, thereby reducing zinc-mediated neuronal injury after brain trauma.

### CROSS-REFERENCE TO RELATED APPLICATIONS

This is a Continuation of U.S. patent application Ser. No. 10/805,882, filed Mar. 22, 2004, which claims the priority benefit of U.S. Provisional Application No. 60/456,392, filed Mar. 21, 2003, all of which are incorporated herein by reference in their entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant No. 1R21NS042882-01A1 awarded the National Institutes of Health.

### FIELD OF THE INVENTION

The present invention relates generally to the field of neuroscience and neurotoxic injuries. More specifically, the present invention relates to controlling zinc release by modulating the synthesis of nitric oxide.

### BACKGROUND OF THE INVENTION

Once regarded as a mere “trace” component of a few odd metalloenzymes, Zn²⁺ is now recognized as a calcium-like messenger, with both transcellular and transmembrane signaling functions. In the cerebral cortex, Zn²⁺ ions are densely concentrated in the presynaptic vesicles of certain glutamatergic neurons. The zinc is released in a calcium- and impulse-dependent fashion during normal physiological activity. This synaptically-released zinc is an established modulator of glutamatergic function, and has been shown to be important for tonic modulation of cortical excitability. Zn²⁺ may also participate in some aspects of synaptic plasticity.

Once regarded as a mere “trace” component of a few odd metalloenzymes, Zn²⁺ is now recognized as a calcium-like messenger, with both transcellular and transmembrane signaling functions. In the cerebral cortex, Zn²⁺ ions are densely concentrated in the presynaptic vesicles of certain glutamatergic neurons. The zinc is released in a calcium- and impulse-dependent fashion during normal physiological activity. This synaptically-released zinc is an established modulator of glutamatergic function, and has been shown to be important for tonic modulation of cortical excitability. Zn²⁺ may also participate in some aspects of synaptic plasticity.

### Synaptically-Released Zinc

Among the transmitters, modulators, and factors released from mammalian neural terminals, one of the more unusual is the cation Zn²⁺, which is both an essential neuromodulator and a potent neurotoxin. In the mammalian forebrain, synaptically-released Zn²⁺ is found exclusively in the presynaptic vesicles of glutamatergic neurons (1). The zinc transporter that concentrates the Zn²⁺ ion in the vesicles (ZnT-3) is expressed in the forebrain only in the zinc glutamate neurons (2) which are a subset of all of the corticopedal glutamatergic fiber systems (3-4). The somata of the forebrain zinc glutamate neurons are all located in cerebral cortex or limbic nuclei, so the zinc glutamate signal is the private neurochemical “voice” of the cerebrocortical and limbic structures. From these neurons there arises a vast, diffuse cortico-cortical-limbic associational network that innervates virtually all of the limbic nuclei and all of the allo- and isocortical regions of the cerebral cortex (5).

When the zinc glutamate neurons are active, they release the Zn²⁺ and the glutamate in a calcium- and impulse-dependent fashion (6-7). Real-time imaging of the Zn²⁺ released during pathway stimulation shows that discrete “puffs” of synaptically-released free Zn²⁺ appear in millisecond and micrometer temporal-spatial regimes. Zn²⁺“puffs” up to 30 μM have been observed (8), representing a 10,000-fold “signal” against the background concentration of free Zn²⁺ in the brain, which is below 3 nM (9).

One physiological function of the synaptically-released Zn²⁺ that is fairly well established is that it modulates the glutamate receptors onto which it is released (10). Practically all GluR and GluM receptors are sensitive, but the net effect of Zn²⁺ in the cerebral cortex seems to be a tonic de-facilitatory, i.e., anti-excitatory, effect. Thus, zinc chelators promote and zinc inhibits epileptiform and paroxysmal activity in many paradigms (11-16). There is also preliminary evidence that synaptically-released Zn²⁺ participates in both developmental (17-18) and experiential synaptic plasticity (19-20).

### Zinc as an Excitotoxin: The Translocation Model

Early clues that zinc might be an endogenous neurotoxin came from a variety of seemingly unrelated observations. It was discovered as early as 1982 that zinc injected directly into the brain was frankly neurotoxic (21). At about the same time synaptic terminals in certain brain regions were found to contain up to 300 μM of releasable free zinc ion (22) and impulse- and calcium-dependent release of that zinc was demonstrated (7). Subsequently, it was shown that the concentration of zinc stored in vesicles, i.e., 300 μM, (23) was enough to kill cortical neurons in culture and shortly there came the serendipitous discovery that neurons could rapidly “dump” their entire 300 μM stores of presynaptic zinc in as little as 1.5 hours of status epilepticus (24-26).

Against this background, the discovery that zinc rapidly disappeared from presynaptic terminals and simultaneously appeared in adjacent postsynaptic neurons in the course of epileptic seizures and convulsions (27) led to the hypothesis that Zn²⁺ was “translocating,” i.e., coming out of terminals and entering postsynaptic neurons, causing or contributing to excitotoxic injury. The neurons that developed zinc positivity are one-for-one the same neurons showing signs of injury, e.g. acidophilia and eosinophilia. Therefore, it was proposed that the translocated Zn²⁺ might be causing the injury, and it was further predicted that zinc chelation might prevent or reduce neuronal injury (27-28).

The simultaneous disappearance of Zn²⁺ from axon terminals and appearance of Zn²⁺ in postsynaptic neurons has since been confirmed in other excitotoxic syndromes, including ischemia (29-30), mechanical brain trauma (31) and various seizure models (32). It has also been repeated in brain slice models of ischemia using simultaneous hypoglycemia and hypoxia (32). Indeed, such translocation of Zn²⁺ from presynaptic terminals into postsynaptic dendrites now appears a likely component of normal, physiological signaling at zinc-modulated synapses (19, 28). Even more importantly from the clinical perspective, zinc chelation is neuroprotective. Elegant experiments showed dramatic “rescue”, i.e., reduction in neurons showing degenerative changes, after ischemia by the simple treatment of intracerebral zinc chelator administration (42).

The concepts of zinc translocation and zinc neurotoxicity have come to be widely endorsed as crucial components of excitotoxic brain injury (2, 34-36). The pathophysiological mechanisms by which excess Zn²⁺ induces apoptosis or necrosis, depending on timing and level, have been elucidated in considerable detail (36-39). Moreover, the use of zinc chelators has been advocated and tested widely as neuroprotectants in excitotoxicity (40-43) and even in degenerative disease (2,44). In general, chelators have proved neuroprotective in stroke and trauma models (31,42), although the results in seizures have been mixed (16,38,45), perhaps because chelation tends to be proconvulsive.

Specific blockade of the high-conductance zinc-permeable channels, the Ca/AK channels, also has given effective neuroprotection (32). The physiological, pharmacological, and toxic levels of rapidly exchangeable zinc in the extracellular fluid of the brain are still eluding exact measurement, but the available information indicates the basal level of free Zn²⁺ is below 3 nM and the toxic level for exposures of a few hours is probably in the single-digit μM range. The level of “free” Zn²⁺ in aqueous habitats that is toxic to invertebrates and even vertebrate fish is also in the 10⁻⁷ M to 10⁻⁶ M range (48).

There have been only two neuroprotection tests with survival of more than a few days. One study found only early neuroprotection in a stroke model (49). The other study found greatly reduced apoptosis in a trauma model using Tunel staining in chelation-protected rats up to 7 days after insult (48).

Beyond excitotoxicity, synaptically released zinc now is recognized as a pivotal factor in the pathogenesis of Alzheimer's disease (50). Daily administration of chelators that bind zinc have been shown to solubilize plaque from the human brain in vitro (51) and transgenic mouse brain in vivo (44) and to yield some cognitive benefits in man as well (52). Most recently, transgenic “Alzheimer's” mice that normally develop amyloid plaques in vivo were shown to develop essentially no plaques when those mice also were lacking synaptically releasable zinc, due to knockout of the vesicular zinc transporter protein, ZnT-3 (53).

### Failures of the Zinc Translocation Model

Despite its continuing heuristic and clinical value, the translocation model is proving inadequate. The pivotal result forcing re-examination of the translocation hypothesis is that neurons acquire bright and vivid staining for zinc in some excitotoxic conditions in which there is simply no demonstrable synaptic, i.e., vesicular, zinc available to be “translocated” into those neurons. This occurs, for example, when the ZnT-3 knockout mouse, lacking detectable vesicular zinc, suffers experimental seizures (54). The same thing happens when a NO* donor is infused directly into the neocerebellum where synaptic zinc is virtually nonexistent (55), which is that the injured neurons turn intensely zinc-positive within minutes.

Perhaps even more revealing is the seemingly paradoxical finding that the intracellular zinc staining produced by both of these procedures, i.e., staining in the cerebellum or in the ZnT-3 knockout, can be blocked by the use of the “classic” extracellular chelator, CaEDTA (56), even when the chelator is delivered after the neurons have become zinc-positive. It appears that this membrane impermeable chelator will effectively “pull” zinc from within previously loaded neuronal somata in vitro and in vivo, even depleting zinc from the vesicles of zinc containing axonal boutons in vivo (56). Presumably this effect of CaEDTA reflects the fact that the chelator irreversibly traps all extracellular zinc, thus preventing all zinc uptake and reuptake, allowing zinc efflux to gradually deplete the intracellular stores.

Other long-standing observations further argue against the translocation hypothesis. For example, it is known that excitotoxicity caused more zinc staining in neuronal somata than in the dendrites of the same neurons, even though the zinc-containing synapses are exclusively on the dendrites, not on the somata (42,57). Also, although zinc chelators give neuroprotection in most paradigms, some failures of zinc chelation are difficult to bring into concordance with the strict translocation model (41).

### Zinc and Excitotoxicity: A New Model

It is proposed that excitotoxicity involves two, possibly three, zinc-release events. The first event is from the presynaptic terminals of zinc-containing neurons, i.e., translocation, and the second is from the zinc-binding proteins of the perikaryal cytoplasm of neurons. Potentially, the third comes from mitochondrial stores in the postsynaptic neurons (37).

Both of the first two events, that is, synaptic release and mobilization of zinc off proteins, can be triggered by NO* infusion into the brain (55, 58) and sustained elevation of NO* and of NO* metabolites, such as peroxynitrite, is a well-established component of the excitotoxic syndrome (59). Conversely, inhibition of NO* synthetase with L-NAME dramatically reduces the number of zinc-positive neurons observed after excitotoxic insult due to blunt-trauma and after pilocarpine-induced seizures. Plausibly, rising NO* drives both synaptic release of Zn²⁺ as well as the mobilization of Zn²⁺ of cytosolic proteins. In brain regions having little synaptic zinc, the mobilization of zinc off proteins can evidently take place independently of any synaptic release, as in the cerebellum or in the ZnT-3 knockout mouse.

On the other hand, in brain regions rich in synaptic zinc, translocation from presynaptic boutons into postsynaptic dendrites or somata almost certainly would take place as originally postulated. After all, the high-capacity zinc-permeable channels Ca/AK and L-type calcium (8,60) would be opened by depolarization, and the zinc concentrations in the clefts would be at least in the mid-micromolar range (8). The recent demonstration that exogenous ⁶⁵Zn penetrates into depolarized neurons during excitotoxicity reinforces the notion of transmembrane Zn²⁺ translocation in excitotoxicity (61). Although zinc generally is thought to inhibit nitric oxide synthase (NOS) (62) in some conditions, toxic influx of zinc evidently activates NOS (63). The latter finding raises the possibility that transynaptic translocated Zn²⁺ could directly activate the NO*-induced mobilization of more Zn²⁺, creating a cytolethal positive feedback loop.

In brain regions with little or no synaptic zinc, the mobilization of zinc from perikaryal metalloproteins would be the predominant event. This would seem to explain that the appearance of zinc staining in neurons after an excitotoxic insult depends on the availability of a metallothionein-III (MT-III) store of “mobilizable” zinc in brain regions with scanty synaptic zinc input and on the presence of zinc-rich terminals, where these are abundant, synapsing on the neurons (49). Of all the zinc metalloproteins, only those with zinc/sulfur coordination environments are susceptible to oxidative mobilization of zinc during excitotoxicity (64). These redox-active sulfur ligands are critical both for very tight binding of zinc and for creation of an environment from which the redox-inert zinc ion can be mobilized by biological oxidants.

Among the thiol-zinc metalloproteins MT-III is a likely source of injury-mobilized zinc. First, MT-III contains 7 metal atoms per molecule (65). Second, it releases zinc atoms in response to NO* more readily than the non-neuronal metallothioneins, MT-I and MT-II (66). This latter sensitivity to NO* is due to MT-III having consensus motifs for catalytic nitrosylation, and that S-nitrosothiols react preferentially with MT-III through transnitrosylation, allowing direct transfer of NO between sulfhydryl groups, thus sequentially releasing zinc ions (66). The fact that glial cells have MT-I and MT-II (66), which are inducible by zinc and can buffer seven atoms of zinc per molecule, whereas neurons lack MT-I and MT-II and have only MT-III (67) potentially explains the preferential vulnerability of neurons to NO*-induced Zn²⁺ toxicity. Again, that mice lacking MT-III show reduced zinc staining after excitotoxic insult (49) directly supports the idea that MT-III is a dominant source of the zinc in injured neuronal somata.

No direct measurements of MT-III concentration in neurons have been made. Generally speaking, MT concentrations in tissue tend to be in the single-digit micromolar range. Assuming neurons have 1.0 μM of zinc, then NO* could liberate 7 μM of free Zn²⁺ in cytosol, a frankly cytolethal dose (68).

With free Zn²⁺ rising to high nM or low μM levels in the cytosol, it would seem likely that Zn²⁺ would start to be “released” from the soma into the extracellular fluid. This might occur when a sufficiently large mobilization of zinc from intracellular proteins occurs, leading to a release of zinc from the resulting zinc-filled somata. This putative “somatic release” could produce extracellular Zn²⁺ signals even in brain regions that have no presynaptic zinc. Zinc effluxers, e.g., ZnT-1 (69), could produce such a release and the presumed zinc influxers could also run backwards when the gradients are extremely high (70). Supporting this idea are recent in vivo microdialysis data showing that Zn²⁺ release in ischemia-reperfusion injury to the brain continues for hours after the presynaptic co-release of glutamate has ceased, suggesting that the continuing release of zinc into the extracellular milieu might be from the neuronal somata (9).

### Zinc Control in Neuroprotective Therapeutic Drugs

Chelation can rescue neurons from excitotoxicity in a number of in vivo and in vitro paradigms. Thus, use of the chelator CaEDTA, which chelates zinc without appreciably affecting Ca²⁺ or Mg²⁺, has proven to be a powerful therapeutic strategy. This strategy has been tested in animal models in several laboratories worldwide with all reporting dramatic, though variable, neuroprotection against excitotoxicity. In a model of blunt-trauma brain impact, the chelator caused a 50%-80% decrease in the number of acidophillic (eosinophilic) neurons after trauma. Overexpression of the zinc scavenger, metallothionein I is also strongly neuroprotective.

However, commonly used heavy metal chelators such as EDTA are polar and thus do not readily penetrate the blood brain barrier. A zinc and copper chelator that, due to its hydrophobicity, passes through the blood-brain barrier is the antibiotic clioquinol. Clioquinol is being used to chelate neuronal zinc in order to dissolve amyloid plaques and thus reduce the pathology of Alzheimer's and Parkinson's disease as disclosed in U.S. Pat. Nos. 5,980,514, 6,001,852, and 6,323,218. Clioquinol potentially could be adapted to treat excitotoxic syndromes, however, serious side effects with clioquinol antibiotic use observed previously in Japan may be problematic for such chelation therapy.

Other researchers in the field are also considering therapeutic or prophylactic approaches which could protect neurons by blocking zinc channels, or which could enhance the brain's capacity to bind and sequester excess Zn²⁺ via delivering excess thionein, histidine, a peptide or a miniprotein, or which could enhance the neuronal zinc efflux pump, ZnT-1. However, preventing the initial release of Zn²⁺ is a more promising therapeutic strategy than trying to “mop up” what has already been released. Currently, the prior art lacks an effective means to inhibit zinc release by targeting molecules that trigger excitotoxic synaptic release of zinc.

### Nitric Oxide Triggers Zinc Release in Neuropathology

Nitric oxide generators infused into the brain cause Zn²⁺ mobilization from cytosolic proteins (41). In addition to mobilizing Zn²⁺ from perikaryal proteins, nitric oxide also caused the disappearance or apparent release of zinc from the axonal boutons. These results, however, are not linked to neuronal injury. Thus, nitric oxide has been shown to release a variety of neurotransmitters and neuromodulators from axonal terminals and boutons. Both calcium-dependent and calcium-independent mechanisms have been identified, and nitric oxide can also inhibit reuptake, thereby further decreasing the amount of release product retained in the boutons.

The notion that nitric oxide can mobilize zinc from intracellular, zinc-binding proteins to produce an anomalous, perikaryal load of “free” zinc fits within the framework of prior work. It has been shown that nitric oxide will oxidize the SH-sites of zinc-binding pockets, thus releasing the Zn²⁺ into intra- or extracellular milieu. This effect of nitric oxide on cellular zinc is apparently independent of the release of Zn²⁺ from axonal boutons, as it occurs even in brain regions such as the neocerebellar cortex that have no zinc in the axonal boutons.

The synthesis of nitric oxide from L-arginine can be inhibited by the L-arginine analogue, L-N-monomethyl-arginine (L-NMMA) and the therapeutic use of L-NMMA for the treatment of toxic shock and other types of systemic hypotension has been proposed in WO 91/04024 and GB-A-2240041. The therapeutic use of certain other nitric oxide synthase inhibitors apart from L-NMMA for the same purpose has also been proposed in WO 91/04024 and in EP-A-0446699.

It has recently become apparent that there are at least three types of nitric oxide synthase as follows: (i) a constitutive, Ca²⁺/calmodulin dependent enzyme, located in the endothelium, that releases nitric oxide in response to receptor or physical stimulation (eNOS); (ii) a constitutive, Ca²⁺/calmodulin dependent enzyme, located in the brain, that releases nitric oxide in response to receptor or physical stimulation (nNOS); and (iii) a Ca²⁺ independent enzyme which is induced after activation of vascular smooth muscle, macrophages, endothelial cells, and a number of other cells by endotoxin and cytokines (iNOS). Once expressed this inducible nitric oxide synthase (iNOS) synthesizes nitric oxide for long periods.

The nitric oxide released by the constitutive enzymes acts as a transduction mechanism underlying several physiological responses. The nitric oxide produced by the inducible enzyme is a cytotoxic molecule for tumor cells and invading microorganisms. It also appears that the adverse effects of excess nitric oxide production, in particular pathological vasodilation and tissue damage, may result largely from the effects of nitric oxide synthesized by the inducible nitric oxide synthase.

The inventors have recognized a need in the art for improvements in treating zinc-mediated neurotoxic injury or insult. The prior art is deficient in methodologies to control or inhibit zinc release during such events by regulating the actions of nitric oxide-stimulated zinc release. The present invention fulfills this long-standing need and desire in the art.

### SUMMARY OF THE INVENTION

The present invention is directed to a method of inhibiting zinc release from neurons. The method comprises providing to the neurons at least one agent that inhibits nitric oxide synthesis and reducing levels of nitric oxide that induces release of zinc from the neurons thereby inhibiting release of zinc therefrom.

The present invention also is directed to a method of preventing zinc-mediated brain injury. The method comprises administering to an individual susceptible to trauma-induced excitotoxicity one or more first agent(s) that inhibits nitric oxide synthesis and reducing nitric oxide-induced release of zinc from neuronal cells in response to the trauma-induced excitotoxicity to prevent the zinc-mediated brain injury. This method further may comprise administering a second agent different from the first agent(s) to improve cerebral blood flow.

The present invention is directed further to a method of improving cerebral blood flow while preventing zinc-mediated brain injury in an individual in need of such therapeutic intervention. The method comprises administering to the individual an agent(s) that inhibits one of or both of neuronal nitric oxide synthase and inducible nitric oxide synthase and administering an agent that increases the activity of endothelial nitric oxide synthase. The combination of the agents modulates nitric oxide synthesis in the individual such that the nitric oxide synthesized improves cerebral blood flow, but does not induce release of neurotoxic amounts of zinc thereby preventing zinc-mediated brain injury.

The present invention also is directed to a related method of improving cerebral blood flow while preventing zinc-mediated brain injury in an individual in need of such therapeutic intervention. The method comprises administering to the individual an agent(s) that inhibits one of or both of neuronal nitric oxide synthase and inducible nitric oxide synthase and, in combination, administering a pressor. The pressor improves cerebral blood flow as the agent(s) reduces nitric oxide-induced release of neurotoxic zinc thereby preventing zinc-mediated brain injury.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 is a schematic showing an overall model of NO*→MT→Zn2+ signaling in the brain. M: metallothionein; NO*: nitric oxide; NOS: nitric oxide synthase (neuronal or epithelial); PDC: protein kinase C; PTP: protein tyrosine phosphatase; Z or Zn: Zn2+.

FIGS. 2A-2B show zinc release by electrical stimulation in the hilus causes an increase in extracellular Zn2+ fluorescence shown in yellow-red in the pseudocolor insets (FIG. 2A) before and after stimulation. Note the rapid rise in the quantitative measure of zinc fluorescence (FIG. 2B).

FIG. 3 shows dialysis recovery and calibration:total process calibration for the EnZin assay. The indicated amount of zinc was added to a “brain”, i.e., a beaker, in which a microdialysis probe was positioned. Dialysate was then collected from the beaker, and the resulting fluorescence spectra were generated upon assaying the dialysate for Zn2+ with the fluorescent EnZin zinc probe. Note that the 2 mM curve is about 10,000 counts higher that the 0 mM curve. Assuming only a 10% recovery, the 2 mM would correspond to 200 nM zinc in the dialysate.

FIG. 4 shows nitric oxide depletes zinc from axonal boutons in vivo. Three hippocampal formation regions, hilus (H), subiculum (Sub) and CA1 were shown stained for vesicular (bouton) zinc with TSQ. Compare normal (FIGS. 4A, 4C, 4E) with that found 2 hr after nitric oxide infusion using spermine NO (FIGS. 4B, 4D, 4F).

FIG. 5 shows nitric oxide depletes vesicular zinc in vivo. Quantitative microfluorimetric analysis shows that vesicular zinc in the hilus was reduced by about 60% compared to control (Cont) by Spermine NO, but unaffected by Spermine alone.

FIG. 6 shows nitric oxide releases Zn²⁺ in vitro. The Zn²⁺ fluorescence in the extracellular fluid of a hippocampal brain slice is normally low (top left) and is raised by addition of the nitric oxide generator SNAP (top right). Subtraction shows the clear Zn²⁺ signal over the zinc-rich boutons in the hilus (lower right). Bright field image shows anatomical landmarks (lower left). The fluorescent “hotspot” is amidst the densest skeins of zinc-rich mossy fibers.

FIG. 7 shows spinal cord crush releases zinc into dialysate. Twelve rats undergoing spinal crush with local microdialysis showed rapid release of zinc into the spinal dialysate. About 1 μM was observed by ICP-mass spectrometry in the dialysate at maximum release.

FIG. 8 demonstrates that NO* controls release from three different cytoarchitectonic regions of hippocampal slices suffering ischemia.

FIGS. 9A and 9B show L-NAME reduces zinc translocation in the hilus (FIG. 9A) and hippocampal cortex (FIG. 9B). Traumatic brain injury (TBI) plus hypoperfusion (hypo) causes zinc translocation, with many zinc-stained neurons identifiable. Prior administration of L-NAME, a non-specific nitric oxide synthesis inhibitor, greatly reduces the number of zinc-stained cells in the TBI⁺ hypoperfusion condition.

FIGS. 10A-10D show cellular co-localization of MT and albumin. HepG2 cells were incubated with F-albumin (Alb) and R-MT, both 250 nM, for 30 min at 37° C. Images are Cys5-labeled concanavalin A, a cell contour marker in blue (FIG. 10A), R-MT in red (FIG. 10B), F-Alb in green (FIG. 10C), and a merged image overlapping all three. Co-localization of MT and albumin is indicated by the yellow and white color in the merged images (FIG. 10D).

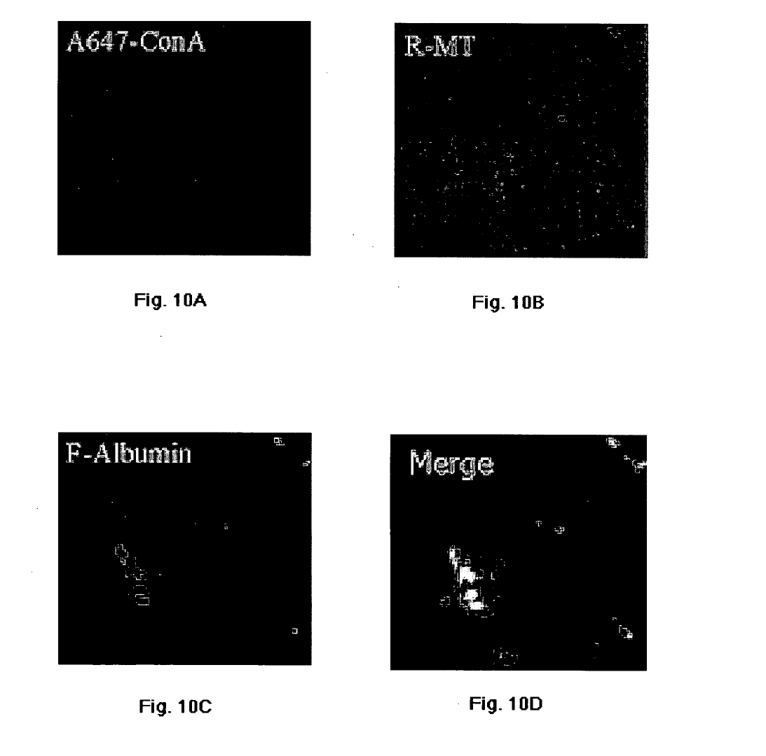

### DETAILED DESCRIPTION OF THE INVENTION

In one embodiment of the present invention there is provided a method of inhibiting zinc release from neurons, comprising providing to the neurons at least one agent that inhibits nitric oxide synthesis; and reducing levels of nitric oxide that induces release of zinc thereby inhibiting release of zinc from the neurons.

In this embodiment the zinc may be located in presynaptic vesicles, in post-synaptic zinc sequestering proteins, or in mitochondrial stores in post-synaptic neurons, or a combination thereof. Additionally, in this embodiment, inhibition of zinc release prevents a zinc-mediated brain injury. The zinc-mediated brain injury may be caused by, for example, stroke, head trauma, ischemia, seizure, or surgery compromising cerebral blood flow.

Also, in this embodiment the agent(s) inhibits the activity of neuronal nitric oxide synthase, inducible nitric oxide synthase or a combination thereof. Representative examples of this agent(s) is 7-nitroindazole, S-methyl-1-thiocitrulline, the protein PIN, 2-amino-5,6-methyl-4H-1,3-thiazine, N(6)-iminoethyl-L-lysine, N(6)-iminoethyl-L-lysine, N-(3-aminomethyl)benzyl acetamidine, or S—[2-amino-(1-iminoethylamino)-5-thioheptanoic acid].

In another embodiment of this invention there is provided a method preventing zinc-mediated brain injury, comprising administering to an individual susceptible to trauma-induced excitotoxicity one or more first agent(s) that inhibits nitric oxide synthesis; and reducing nitric oxide-induced release of zinc from neuronal cells in response to the trauma-induced excitotoxicity thereby preventing zinc-mediated brain injury.

This embodiment further comprises the method step of administering a second agent to improve cerebral blood flow where the second agent is different from the first agent(s). The second agent may increase the activity of endothelial nitric oxide synthase. Representative examples are simvastatin, 17-beta-estradiol, a corticosteroid, endothelin, or AT2 receptor agonists. An example of a corticosteroid is dexamethasone. Alternatively, the second agent may be a pressor. Examples of a pressor are dopamine, vasopressin, angiotensin II, or epinephrine.

In all aspects of this embodiment the first agent may inhibit nitric oxide synthetic activity of neuronal nitric oxide synthase, of inducible nitric oxide synthase, of both neuronal nitric oxide synthase and inducible nitric oxide synthase or a combination thereof. Examples of a first agent are 7-nitroindazole, S-methyl-1-thiocitrulline, the protein PIN, 2-amino-5,6-methyl-4H-1,3-thiazine, N(6)-iminoethyl-L-lysine, N(6)-iminoethyl-L-lysine, N-(3-aminomethyl)benzyl acetamidine, or S—[2-amino-(1-iminoethylamino)-5-thioheptanoic acid]. Additionally, the first and second agents and the pressor may be administered in a pharmaceutical composition comprising a pharmaceutically acceptable carrier.

Furthermore, in all aspects of this embodiment release of the excitotoxic zinc is caused by stroke, head trauma, ischemia, seizure, or surgery compromising cerebral blood flow. Representative examples of surgery are cardiaobypass, cardiopulmonary bypass or carotid endarterectomy. Additionally, the excitotoxic zinc may be released from presynaptic vesicles, post-synaptic zinc sequestering proteins or mitochondrial stores in the post-synaptic neurons.

In a related embodiment there is provided a method of improving cerebral blood flow while preventing zinc-mediated brain injury in an individual in need of such therapeutic intervention, comprising administering to the individual an agent(s) that inhibits one of or both of neuronal nitric oxide synthase and inducible nitric oxide synthase, and administering an agent that increases the activity of endothelial nitric oxide synthase, where the combination of the agents modulates nitric oxide synthesis in the individual such that the nitric oxide synthesized improves cerebral blood flow, but the nitric oxide does not induce release of neurotoxic amounts of zinc thereby preventing zinc-mediated brain injury.

In all aspects of this embodiment the agents inhibiting nNOS or iNOS, the agent increasing activity of eNOS and the pharmaceutical compositions thereof and the locations from which neurotoxic zinc is released are as described supra. Also, in all aspects the zinc-mediated brain injury is caused by stroke, head trauma, ischemia, seizure, or surgery that would compromise cerebral blood flow. The surgeries are described supra.

In another related embodiment there is provided a method of improving cerebral blood flow while preventing zinc-mediated brain injury in an individual in need of such therapeutic intervention, comprising administering to the individual an agent(s) that inhibits one of or both of neuronal nitric oxide synthase and inducible nitric oxide synthase; and in combination, administering a pressor where the pressor improves cerebral blood flow as the agent(s) reduces nitric oxide-induced release of neurotoxic zinc thereby preventing zinc-mediated brain injury.

In all aspects of this embodiment the agents inhibiting nNOS or iNOS, the pressor and the pharmaceutical compositions thereof and the locations from which neurotoxic zinc is released are as described supra. Also, in all aspects the zinc-mediated brain injury is caused by stroke, head trauma, ischemia, seizure, or surgery that would compromise cerebral blood flow. The surgeries are described supra.

As used herein, the term “zincation” refers to Zn²⁺ binding to a protein.

Provided herein are methods of interrupting or influencing the NO*→MT→Zn²⁺ signaling pathway to inhibit release of zinc from neurons to reduce or prevent zinc-mediated brain injury, such as brain injury associated with stroke, ischemia, seizures or traumatic head injury that involve “excitotoxicity” due to excessive release of Zn⁺² from neurons. In general, excitotoxic zinc can be released from presynaptic vesicles, post-synaptic zinc sequestering proteins such as metallothioneins and mitochondrial stores in the post-synaptic neurons. Immediate therapeutic applications of modulators of this pathway can be beneficial during or after, inter alia, stroke, trauma, ischemia/reperfusion, seizures, or, as prophylaxis, prior to surgery compromising cerebral blood flow.

Furthermore, it is contemplated that the same NO*→MT→Zn²⁺ cascade that is activated in excitotoxicity is also activated to a weaker degree in the course of normal, physiological brain activity and is a key part of normal glutamatergic synaptic function. For example, in the particular case of stimulation-induced synaptic plasticity (long term potential; LTP) zinc is mobilized in a way similar to, though weaker than, in excitotoxic injury. Moreover, the evidence indicates that the zinc signals in this LTP paradigm are an essential part of the synaptic plasticity. Thus, it is likely that the same NO*→MT→Zn²⁺ signals that mobilize zinc in injury also mobilize zinc in the LTP situation and likely in other normal function as well.

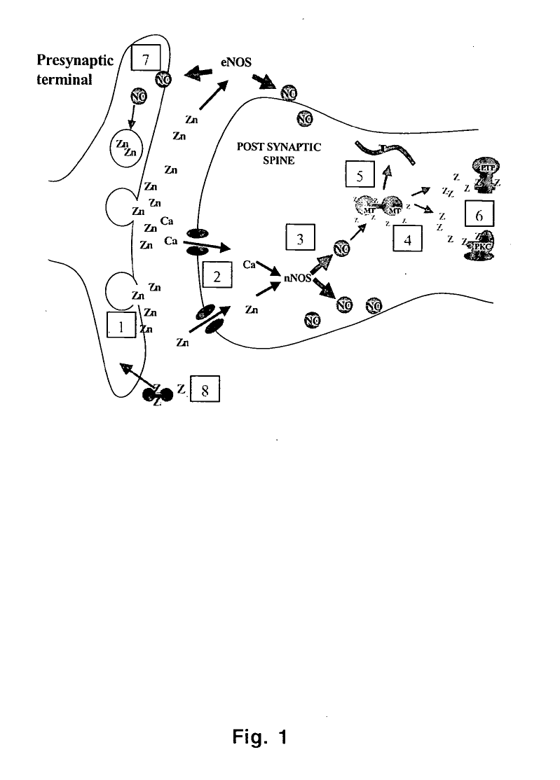

The major components of the NO*→MT→Zn²⁺ pathway as depicted in FIG. 1, that can be monitored directly are 1) presynaptic zinc release, both as a decrease in vesicular Zn²⁺ and as an increase in extracellular Zn²⁺, 2) Zn²⁺ rising in the cytoplasm of postsynaptic neurons, 3) generation of NO*, 4 & 5) changes in the zincation of MT, the zinc chaperone, as either a gain or loss of Zn²⁺, 7) NO*-induced release of synaptic zinc, and 8) MT-mediated reuptake of zinc from the extracellular fluid. Steps 2 and 4 both result in increased Zn²⁺ in the postsynaptic cytosol and cannot be directly discriminated. They may be indirectly discriminated by, for example, using MT-3 knockouts or by blocking Zn²⁺ channel opening.

Accordingly, zinc release from neurons can be modulated through regulation of nitric oxide synthesis via the nNOS, iNOS and eNOS enzymes. Modulation of zinc release encompasses using therapeutic agents that can either inhibit or activate these enzymes during an excitotoxic event. Production of NO* after neurotoxic injury or insult can be both harmful in inducing release of zinc from presynaptic vesicles and from post-synaptic zinc sequestering proteins and mitochondrial stores in post-synaptic neurons or beneficial in augmenting cerebral blood flow. Additionally, in that the NO*→MT→Zn²⁺ pathway may function during normal physiological status, it is contemplated further that therapeutics addressing the NO*→MT→Zn²⁺ pathway may include signal amplifiers for some conditions, as well as the signal blockers.

Nitric oxide synthesis inhibitors have been described in the art (75). For example, WO94/12165, WO94/14780, WO93/13055, EP0446699A1 and U.S. Pat. No. 5,132,453 disclose compounds that inhibit nitric oxide synthesis and preferentially inhibit the inducible isoform of nitric oxide synthase. Other examples of nitric oxide inhibitors may be obtained from U.S. Pat. Nos. 6,465,518, 6,462,044, 6,448,286, 6,410,542, 6,344,483, 6,344,473, 6,169,089, 6,143,790, 6,136,829, 6,071,906, 6,043,261 and 6,011,028.

Nitric oxide synthesis inhibitors may be selective for nNOS, iNOS and may be used individually or in combination to reduce zinc-mediated brain injury. The most commonly studied selective nNOS inhibitor is 7-nitroindazole (7-NI). Other selective nNOS inhibitors include S-methyl-1-thiocitrulline (SMTC) and the 89 amino acid, 10 kDa protein inhibitor of nNOS (PIN). Representative examples of selective inhibitors of iNOS include, but are not limited to, 2-amino-5,6-methyl-4H-1,3-thiazine (AMT), N(6)-iminoethyl-L-lysine, N-(3-aminomethyl)benzyl acetamidine (1400 W) and S—[2-amino-(1-iminoethylamino)-5-thioheptanoic acid] (GW274150).

Additionally, it may be beneficial to enhance the activity of eNOS to improve cerebral blood flow while inhibiting the activities of nNOS and/or iNOS to prevent zinc-mediated brain injury. eNOS activators include, but are not limited to, simvastatin, 17-beta-estradiol, corticosteroids, such as dexamethasone, the protein endothelin (ET-1) and AT2 receptor agonists. Note that the nitric oxide synthase inhibitors, L-NAME (N(6)-nitro-L-arginine methyl ester) and L-NAA (N-omega-amino-L-arginine) are non-selective nitric oxide synthase inhibitors in that they inhibit all three nitric oxide synthase isoforms and may cause problems therapeutically in that inhibition of eNOS may reduce blood flow. Alternatively, a pressor, e.g., the α- and β-adrenergic catecholamine dopamine (83) may be used in combination with nNOS and/or iNOS to augment cerebral blood flow while simultaneously reducing zinc-mediated brain injury.

The NOS inhibitors, NOS activators and pressors of the instant invention may be used to reduce zinc-mediated brain injury which involves “excitotoxicity” due to excessive release of Zn⁺² from neurons, for example, as a result of neurotoxic injury, seizure or stroke, while simultaneously augmenting cerebral blood flow (CBF) in brain injury associated with cerebral ischemia, such as traumatic head injury, or prophylactically in surgeries, e.g., cardiobypass, cardiopulmonary bypass and carotid endarterectomy. In reducing zinc-mediated brain injury NOS inhibitors may include, but are not limited to, the nNOS inhibitor 7-nitroindazole (7-NI) and the iNOS inhibitor 2-amino-5,6-methyl-4H-1,3-thiazine (AMT). In augmenting cerebral blood flow in secondary ischemic brain injury or during surgery the eNOS activator may the corticosteroid dexamethasone and the preferred NOS inhibitors are the nNOS inhibitor 7-nitroindazole (7-NI) and the iNOS inhibitor 2-amino-5,6-methyl-4H-1,3-thiazine (AMT). Alternatively, a pressor, such as, dopamine, vasopressin which is a natural nonapeptide hormone secreted from the posterior pituitary, angiotensin II which is an octapeptide or epinephrine, may be used instead of an eNOS activator.

The NOS inhibitor(s), alone or in combination with eNOS activator or a pressor, may be administered as a composition comprising a pharmaceutically acceptable carrier compatible with these compounds and any reagents necessary for administration. In preparing such a composition, any conventional pharmaceutically acceptable carrier may be utilized. Typical carriers for administration by injection would be sterile aqueous/buffered solutions, preferably water for injection or unbuffered or buffered physiological saline. The NOS inhibitor(s), the eNOS activator or the pressor are each preferably present in the carrier at a concentration of 1 mM to 300 mM, especially from 50 mM to 200 mM.

In carrying out the methods of the present invention, the NOS inhibitors and/or the eNOS activators and/or the pressors may administered to patients daily in an amount from about 1 mg/kg to about 100 mg/kg in single or divided doses or continuously through chronic pump infusion (61-74). The preferred dosage will vary depending upon the clinical indication presented by the type of zinc-mediated brain injury. For treatment of patients who have suffered an acute neurotoxic insult, the dosage may be about 5 mg/kg to about 50 mg/kg daily. The duration of treatment for acute neurotoxic conditions may be from 1 to about 7 days. For treatment of patients who suffer from chronic neurotoxic conditions a dosage of about 1 mg/kg to about 10 mg/kg daily may be used.

Administration may be carried out by injection or by infusion utilizing a chronic infusion pump of a pharmaceutical composition, comprising the NOS inhibitor(s) and/or NOS activator(s) and/or pressor(s) into the cerebrospinal fluid of the patient. For example administration may be into the lateral ventricles or lumbar sac. Injection can also be by intraperitoneal or intravenous routes.

The following examples are given to illustrate various embodiments of the invention and are not meant to limit the present invention in any fashion.

### Example 1

### NO* Induces Neuronal Zn²⁺ Release

### Methods

### Brain Slice Preparation

Experiments were performed on transverse hippocampal splices taken from 5 to 6 week-old male rats. In all, 41 hippocampal slices and 10 additional hippocampi taken directly from intact animals (“freshly frozen” see below) were studied quantitatively. After halothane (3.5%) inhalation in the anesthetic box, the rats were perfused with 4° C. artificial cerebrospinal fluid (ACSF) (NaCl was substituted by 190 mM sucrose) intracardially for 1 minute (76-77).

After perfusion, the rats were decapitated and the hippocampi were dissected out and placed in cold (4° C.) artificial cerebrospinal fluid. Transverse slice (400 μm) were cut with a vibratome (Campden Vibraslice, model # 752) and placed on a nylon mesh in an incubation chamber. The artificial cerebrospinal fluid temperature in the incubation chamber was then gradually increased from 4° C. to 20° C. for >30 minute. The slices were kept at 20° C. in oxygenated (95% O₂, 5% CO₂) artificial cerebrospinal fluid for 30 minute before stimulation. The gas mixture was also bubbled through the water bath in the chamber and vented over the chamber for >30 minute before the experiment started. The standard artificial cerebrospinal fluid comprised 130 mM NaCl, 3.5 mM KCl, 1.25 mM NaH₂PO₄, 2 mM MgSO₄, 2 mM CaCl₂, 20 mM NaHCO₃, 2 mM ascorbic acid and 10 mM D-glucose.

### Fluorescence Microscopy of Zinc Release

Because ACS reagent grade Mg²⁺ and Ca²⁺ salts contain significant levels of Cu²⁺ and Zn²⁺ as impurities, the phosphate-buffered saline was prepared by first combining all ingredients but the calcium and magnesium salts and passing the buffer through a Chelex-100 column (Bio-Rad, Richmond, Calif.) to remove metal ion impurities, then adding Suprapure grade CaCl₂.4H₂O (catalog no. 2384-2, EM Science, Gibbstown, N.J.) and Puratronic grade MgSO₄.7H₂O (catalog no. 10801, Alfa AESAR, Ward Hill, Mass.) to the appropriate concentration. Brain slices were placed on the thermoregulated stage of an Olympus IX70 inverted microscope.

For measuring zinc release with EnZin, (apoCA+ABDN), the slices were epi-illuminated with 420 nm light and imaged through both 530 nm and 700 nm band pass filters. EnZin is a mixture of the zinc-binding protein, carbonic anhydrase, from which the zinc has been removed (hence, “apo”CA) and a fluorescence indicator, ABDN, which was made by a one-step synthesis from ABDF (Molecular Probes). The ABDN is fluorescent at 600 nm when free, but fluoresces at 560 nm when bound to the Zn+CA (“holo” CA). Thus, the CA is the zinc detector, and the ABDN is the ratiometric fluorescent reporter. A variety of CA with different zinc affinities for different zinc measurement ranges can be generated (78).

The ratio of the 520/700 nm images were then calculated (Optimus), giving a ratiometric image of zinc concentration. In this fluorescence system, the affinity of the EnZin is so high (Kd=4 μM) that it can be treated as infinite. With a stoichiometric excess of EnZin, the percent occupancy of the EnZin by Zn²⁺ can be used as the modulator of fluorescence intensity. In this way, a dynamic range of about 500-fold of Zn²⁺ concentrations, ranging from 100% saturation of the EnZin to about 0.05% saturation was obtained. To be explicit, with 20 μM of the apoCA, the dynamic range is from about 10 nM to 20 μM. With a 420 nm excitation band pass filter, 500 nm dichroic, and a 540 nm emission band pass filter, images were collected through a 10×0.4 NA UplanApo Olympus objective and imaged with a Diagnostic Spot cooled megapixel CCD camera and Spot Image analysis software.

Alternatively, for faster imaging and quantitation (albeit with lower resolution), a NORAN Odessey laser scanning confocal (Nikon Inverted microscope) can be used. This microscope is equipped with a HeCd laser (420 nm) especially chosen for the EnZin excitation, and can collect both the 560 and the 600 nm fluorescence simultaneously for real-time ratiometric imaging of zinc release.

### Intrahippocampal and Intracerebellar Injection of Nitric Oxide Donor

Adult Sprague-Dawley rats (male, 300-350 g) were anesthetized with halothane, and mounted in a stereotaxic instrument where a bone defect was opened after reflection of the scalp. A dialysis probe was stereotaxically-lowered into the hippocampus (4.0 mm caudal to bregma, 2.5 mm lateral, 3.2 mm subdura, N=9) and cerebellar cortex (5.0 mm caudal to lambda, 2.5 mm lateral to midline, 1.5 mm below the dura, N=2). Infusions through the dialysis probe included either a nitric oxide donor (Spermine-NONOate, 100 mM/2 μL, Molecular Probes, Eugene, N=3), a negative control (saline, 0.9% NaCl, 2 μL, N=3), or a sham donor (Spermine, Molecular Probes, 100 mM/2 μL, N=3) delivered over 5 minutes into the hippocampus.

### Dialysis Methods

The microdialysis procedures that allow continuous sampling of the brain's extracellular fluids have been described previously (79). Briefly, artificial cerebrospinal fluid containing 147 mM NaCl and 4.0 mM KCl was run through a Chelex 100 ion exchange column to remove all divalent cations, then 2.3 mM CaCl₂ and 0.9 mM MgCl₂ (Ultrex, ultrapure) were added to make a very low-Zn²⁺ artificial cerebrospinal fluid. Direct assays of Zn²⁺ in this artificial cerebrospinal fluid indicate that there was less than 10 nM of Zn²⁺.

This fluid was pumped through a 4 mm length of dialysis tubing (cutoff=20 KD) that has been stereotaxically-placed in the brain region of interest. Pumping rate is 2 μL per min. Efflux from the dialysis catheter was collected in 10-40 μL aliquots and analyzed for nitric oxide with a Severs Model 280 m NO analyzer, for glutamate by HPLC, and for zinc by the method of EnZin fluorimetry. The dialysis tubing and connecting tubing were all washed by pumping a dilute EDTA solution through before use. This technique gives high spatial and temporal resolution of nitric oxide, glutamate, and zinc release during and after the traumatic brain injury and any subsequent experimental hypoperfusion.

### EnZin Fluorimetry for Zinc

Twenty μL aliquots of the diaylsate were taken without further modification and added directly to the EnZin solution at a ratio of 20 μL:110 μL. This 130 μL was pipetted into a 200 μL capacity quartz cuvette which was placed in a SPEX Fluorolog spectrofluorometer. With an excitation beam or 420 nm±5 nm, emissions were collected at 2 nm intervals (2 sec integration) across the spectrum of 425 nm to 700 nm.

Two methods were used for calibration standard: simple calibration and the method of standard additions. In the first method, the 20 μL aliquot of dialysate was replaced with 20 μL aliquots of DI with known amounts of ZnCl₂ added. For the standard addition, the ZnCl₂ solutions was mixed with dialysate, and 20 μL aliquots of the mixtures were added to the EnZin solution. Final quantitation was done using either “center of gravity” software to quantify shift in emission maxima or using simple ratios of HoloCA-ABDN peak emission (560 nm) to apoCA-ABDN emission (600 nm).

The EnZin solution consists of 20 μM of bovine CA I (stripped of its zinc by established “apo-izing” methods (75), and 30 μM of the fluorescent indicator ABDN. In this system, the apoCA chelates any free Zn²⁺ quantitatively (K_{D}=4 μM), and the ABDN then associates quantitatively with the Zn²⁺-bound CA (K_{D}=45 μM). HoloCA-bound ABDN fluoresces at 560 nm; free ABDN and apoCA-bound ABDN fluoresces at 600 nm.

### Zinc Measurements by TSQ Quantitative Microscopy

For measuring the normal concentration of vesicular zinc in the tissue after treatment with various nitric oxide generators, frozen tissue sections were cut in a closed-top cryostat at 30 μm (−14° C.). For whole brains, starting at 3.6 mm caudal to bregma, every 3rd section was saved until 5-10 sections were obtained. For tissue slices studied in vitro, every section was saved (typically 6-9 per brain slice). After cutting, the sections were thawed onto glass slides, dried, then stained with TSQ (80-82).

### TSQ Fluorescence Staining

The sections were stained with TSQ, ((N-(6-methoxy-8-quinolyl)-para-1-toluenesulfonamide, Molecular Probes), as described previously (80) for fluorescence visualization of Zn²⁺. Briefly, the frozen, unfixed sections were immersed in a solution of TSQ (4.5 μM) in 140 mM sodium barbital and 140 mM sodium acetate buffer (pH 10-10.5) for 60 seconds, rinsed for 60 seconds in normal saline (NaCl, 0.9%), then viewed and imaged in the microscope with no coverslip while still damp. A SPOT2 cooled CCD camera was used to capture the fluorescence images (10×n.a. 0.65) at 12 bit resolution for subsequent quantitation of zinc-TSQ fluorescence intensity. Control and treated tissues were always digitized in the same sitting to guard against sensitivity changes in the system.

### Head Trauma and Hypoperfusion Methods

Male Sprague-Dawley rats weighing 350-400 g were anesthetized with isoflurane in an anesthetic chamber, intubated, and mechanically ventilated with 1.5-2.0% isoflurane in O₂:room air (70:30) using a volume ventilator (EDCO Scientific, Chapel Hill, N.C.). Polyethylene cannulae were placed in both femoral arteries and one femoral vein for arterial pressure monitoring and hemorrhage and for drug infusion, respectively. Rectal and temporalis muscle temperatures were monitored using telethermometers (Yellow Springs Instruments, Yellow Springs, Ohio). Rectal temperature was maintained using a thermostatically controlled water blanket (Gaymar, Orchard Park, N.Y.).

Rats were prepared for parasagittal fluid-percussion traumatic brain injury (TBI) and for laser Doppler flowometry as described below. Intracranial pressure (ICP) was monitored continuously using a Camino probe (V420, Camino Laboratories, San Diego, Calif.) placed in a lateral ventricle through a small craniotomy placed posterior and lateral to the fluid percussion injury site. Isoflurane was lowered to 1.5%; the rats were connected to the trauma device, and subjected to moderate or severe traumatic brain injury.

After traumatic brain injury (or sham-TBI) blood was withdrawn from a catheter in a femoral artery to reduce MAP to 60 mmHg. Mean arterial pressure was maintained at 60 mmHg for 45 minutes during which time cerebral blood flow and ICP were measured. After 45 minutes of hypotension, the test fluids were infused. Post-resuscitation measurements were then performed. An arterial blood sample was withdrawn with each measurement series to determine PaCO₂, PaO₂, pH and hemoglobin concentration or hematocrit.

### Fluid-Percussion Traumatic Brain Injury (TBI)

Fluid-percussion traumatic brain injury is a clinically relevant model that has been used in many studies of traumatic brain injury in cats, rats, and pigs (79, 82). The trauma device consists of a Plexiglas cylinder 60 cm long and 4.5 cm in diameter. One end of the device is connected to a hollow metal cylinder housing a pressure transducer (Statham PA856-100) and the other end is closed by a Plexiglas piston mounted on O-rings.

The transducer housing is connected to the rat by a hollow metal tube, cemented to a craniotomy trephined in the skull immediately lateral to the superior sagittal sinus midway between bregma and lambda. The piston is struck by a 4.8-kg steel pendulum dropped from a variable height, which determines the intensity of injury. The pressure pulse is recorded on a storage oscilloscope triggered photoelectrically by the descent of the pendulum. Sham-injured rats were connected to the trauma device but were not injured.

The effects of a moderate (2.0 atm) or severe (3.0 atm) levels of injury were studied. Moderate injury (2.1 atm) is associated with 10% mortality, reductions in EEG amplitude for 10 to 15 min, brief (<5 min) suppression of reflexes (corneal, pinna, paw flexion to pinch, righting), and return of spontaneous locomotion within 10 min. Severe TBI (2.9-3.4 atm) is associated with a 22 to 40% mortality rate, suppression of corneal, righting and paw flexion reflexes for 10-20 min and suppression of spontaneous locomotion for 30-40 min.

### Measurement of Nitric Oxide Levels

Nitric oxide levels were measured in vivo using an electrically isolated nitric oxide potentiostat (ISO-NO Mark II, World Precision Instruments, Inc., Sarasota, Fla.) with a 30 μM shielded sensor. The ISO-NO system, in which nitric oxide oxidizes platinumized carbon fibers to produce an electrical current, is calibrated daily prior to each experiment using nitric oxide donors (e.g., SNAP, S-nitroso-N-acetyl-L-penicillamine). The amperometric probes are insensitive to nitrite, hydrogen peroxide, L-arginine and changes in CO₂, O₂ and pH. The fragile carbon fiber probes were inserted into the cerebral cortex through a guide cannula advanced through a small craniotomy posterior to the fluid percussion injury site.

Mean arterial blood pressure, cerebral blood flow, neurological scores and numbers of surviving neurons are considered the primary endpoints of these studies. Mean arterial pressure and cerebral blood flow were compared among groups using analysis of variance for a two-factor experiment with repeated measures on time after resuscitation. The two factors were group (7.5% saline, shed blood, hypertonic L-arginine) or time after resuscitation. Post-hoc analyses were performed using Bonnferroni/Dunn tests. Neuron counts can be compared among groups using a one-factor analysis of variance. Neurological scoring yields ordinal data which can be compared among groups using Kruskall-Wallis procedures.

The secondary endpoints SVR, ICP, arterial blood gases, NO and O₂⁻ levels also can be compared using a repeated measures analysis of variance with factors of group and time. The scoring system for the nitrotyrosine immunoreactivity yields ordinal data, which can be compared among groups using Kruskall-Wallis procedures.

### Zinc Detection Using Newport Green Fluorescence Microscopy

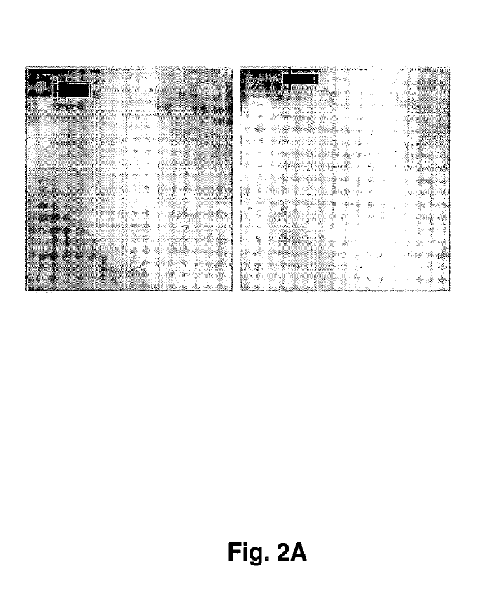

Brain slices were incubated with the extracellular fluorescent zinc indicator Newport Green (cell impermeable), and the slices were then stimulated with brief bursts of bipolar electrical stimulation (5 sec, 100 Hz, 0.1 msec) delivered to the cell bodies (granule neurons) of the zinc-rich mossy fiber axons (FIG. 2A). Upon stimulation, there was an abrupt onset of green fluorescence in the extracellular fluid, indicating the release of zinc. Although Newport Green is not ratioable, semi-quantitative estimates of the zinc release in these studies indicated that the zinc levels easily reached 30 μM in the fluid. Note also the speed of release. In the current system, a zinc “flash” can be detect after just 3 (30 msec) pulses at the start of a 100 Hz stimulus train (FIG. 2B).

### Zinc Detection Using EnZin Fluorescence Microscopy

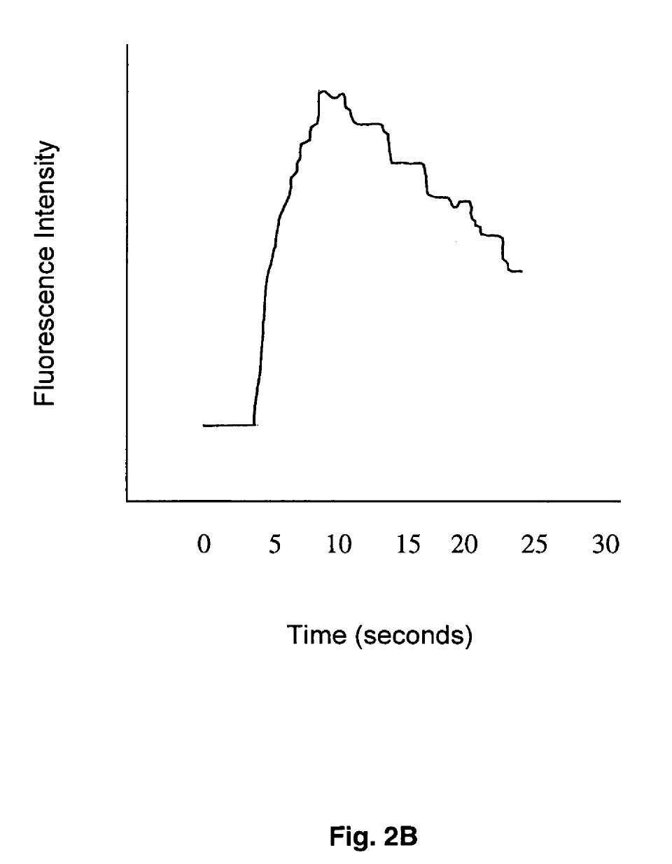

EnZin was developed (78) for the purpose of high-accuracy, ratiometric determinations of Zn²⁺ in biological systems. The microdialysis procedures that allow continuous sampling of the brain's extracellular fluids have been described above (79). The EnZin fluorescent method can be used for assaying only the weakly bound Zn²⁺ in dialysates. The ratiometric zinc fluorescent indicator EnZin can detect as little as 10 μM of Zn²⁺. In the initial tests of the dialysis approach, it was verified that the EnZin assay can easily detect <<2 μM of Zn²⁺ in a phantom brain (beaker of saline). In those tests, the zinc was assayed from the dialysate (not the beaker), so the sensitivity refers to zinc levels in the brain (FIG. 3).

In direct tests of sensitivity in the dialysate, it was found that the assay can discriminate 50 nM from 100 nM with ease. Therefore, with recoveries of, say 20%, the EnZin assay can easily detect brain zinc release concentrations in the range of 250 nM. Considering in vitro data have indicated at least 30 μM of Zn²⁺ was released from brain slices despite the low-release capability of slices in general, this level of detection capability should be more than adequate for zinc release measurement in vivo.

### Example 2

### NO* Induces Neuronal Zn²⁺ Release In Vivo and In Vitro

### Depletion of Zinc From Axonal Boutons In Vivo

Rats were anesthetized, a microsyringe and needle were lowered into the hippocampal formation or neocerebellar cortex by stereotaxis and then nitric oxide generators were infused into the tissue. After 120 minutes of survival, the rats were sacrificed and the brains were cut and stained to show the location and concentration of the weakly bound Zn²⁺ that is normally sequestered in the presynaptic boutons, i.e., vesicular zinc. Zinc localization and quantification were determined by TSQ fluorescence staining.

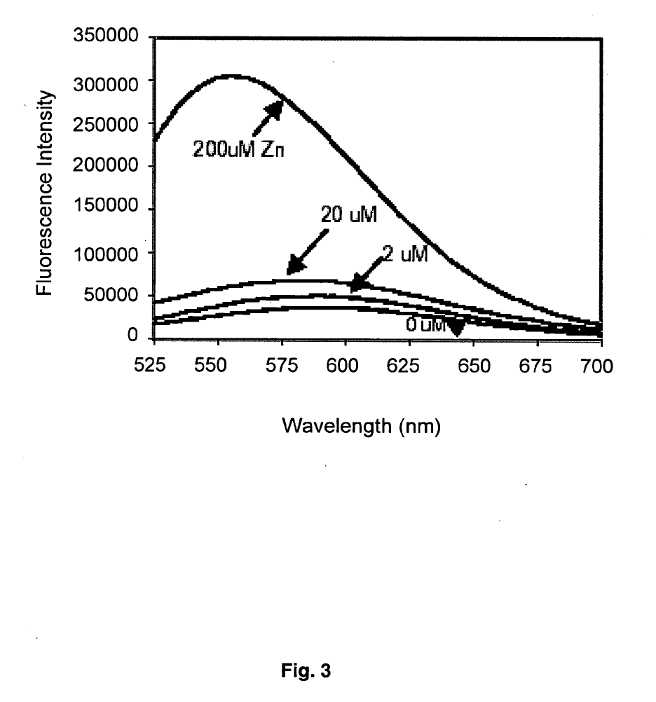

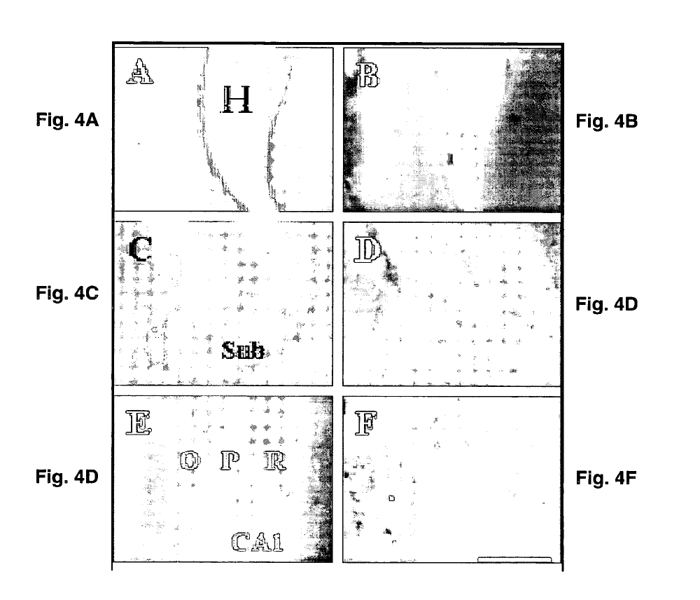

The results were the same for all brain regions that were innervated by zinc-containing axonal projections and were within reach of the nitric oxide. As shown in FIGS. 4A-4F and in FIG. 5, the infusion caused virtually a complete loss of the vesicular zinc staining from the boutons. Prior work has shown that the only source of “stainable” zinc in normal brain tissue is the zinc in the vesicles of the zinc-containing axonal boutons. Thus, the loss of fluorescence shown in FIGS. 4A-4F and in FIG. 5 can be accepted without doubt as a loss of the pool of weakly bound Zn²⁺ that is normally sequestered in the presynaptic vesicles.

Time Course of Bouton Depletion after Nitric Oxide Infusion In Vivo

It is believed that the Zn²⁺ release after nitric oxide is very fast, perhaps occurring within minutes of nitric oxide arrival. This is because virtually complete elimination of all “stainable” Zn²⁺ took place in the boutons within just 2 hours of nitric oxide generator infusion (FIGS. 4B, 4D, 4F). If the effect is as fast as it is suspected, this would be important both theoretically (concerning release mechanism) and clinically (concerning the therapeutic window of opportunity). For this reason, the time course of zinc release can be examined by in vitro studies that involve sacrificing rats at various intervals after nitric oxide infusion. Direct real-time monitoring of Zn²⁺ release from hippocampal brain slices can further be determined by EnZin fluorescence analysis.

Release of Zn²⁺ into Extracellular Milieu In Vitro

The loss of staining does not necessarily mean the actual loss or release of the Zn²⁺. Conceivably, what nitric oxide does in boutons is to change the binding state of the zinc from weakly bound (stainable) to tightly bound. This would cause the Zn²⁺ to “disappear” from staining without actually being released. This has not been proven to be the case in any of the half a dozen or so paradigms in which loss of staining has proved to be due to synaptic release. It is important, however, to test directly for release of Zn²⁺ into the extracellular milieu by nitric oxide.

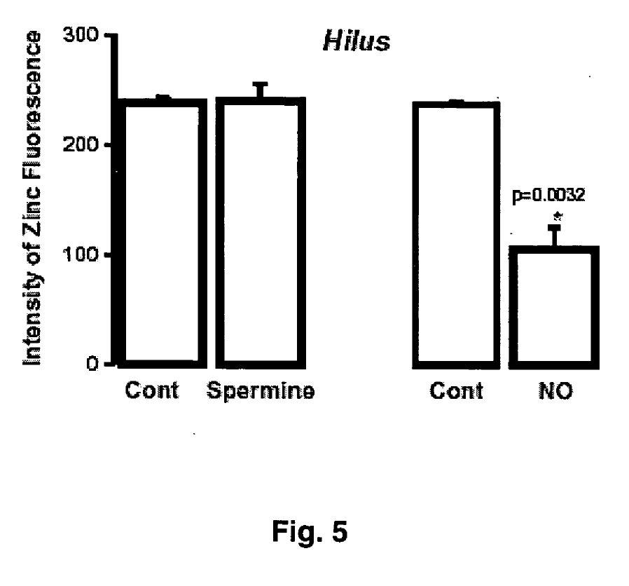

Acute hippocampal brain slices were prepared and maintained conventionally as described (83-84). Results from the EnZin assay showed a rapid (within seconds) increase in the Zn²⁺ fluorescence in the slice bath immediately after adding nitric oxide generators SNAP (FIG. 6) or Spermine NO. Spermine alone had no effect. Taken together with the loss of zinc staining in the boutons after nitric oxide infusion (FIGS. 4B, 4D, 4F), this rapid appearance of Zn²⁺ in the extracellular milieu after nitric oxide is virtually certain to be reflective of release of Zn²⁺ from the boutons.

Timing of Zn²⁺ Release into Extracellular Space

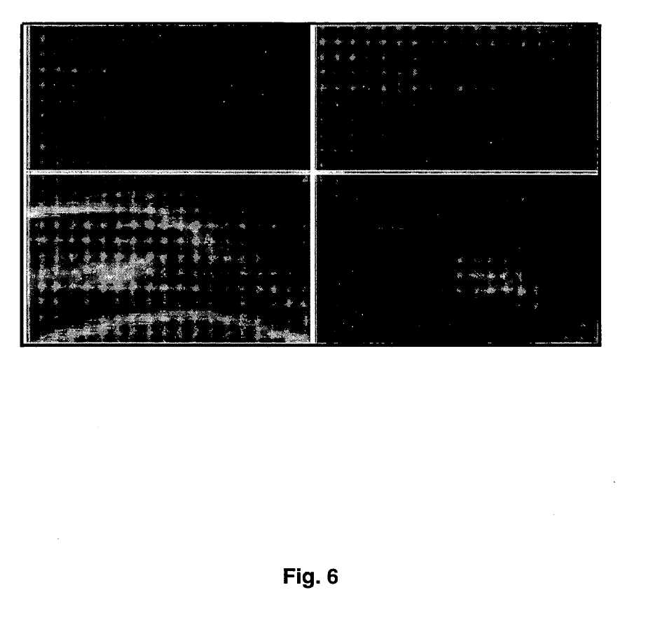

Microdialysis method, which can monitor zinc release from a to-be-injured brain site before, during, and after the injury, was used to determine when the zinc is released after neural injury. The studies were first applied to the case of traumatic injury of the spinal cord. The spinal cord has only very sparse zinc innervation, so the role of zinc in spinal injury would be expected to be rather small. Nonetheless, when dialysis samples were taken from the rat spinal cord before and after a transient “crush”, a substantial release of zinc was observed that began almost immediately and continued for about 3 hours (FIG. 7). The timing of Zn²⁺ release into the extracellular space of brain tissue after stroke, head trauma, or ischemia is vital to know because it will dictate the timing of effective therapeutic intervention, i.e., the “zinc therapy window.”

### Characterization of Zinc Release Induced by Nitric Oxide

Data presented above indicate that nitric oxide depletes Zn²⁺ from neuronal boutons in vivo and causes a complementary release of Zn²⁺ from brain slices in vitro. Further experiments may be done to confirm nitric oxide induces zinc release from boutons. Specifically, in the brain slice experiments, it is important to verify that the Zn²⁺ efflux is coming from boutons via vesicular exocytosis and not being released into the superfusate from other cellular compartments such as neuronal or glia perikarya. In addition, while the observed depletion of boutons in vivo appears to be the same release that was observed in vitro, it is important to verify there is genuine release into the extracellular fluid in the intact brain. This is vital because it is only by such release that boutons can induce zinc-mediated neurotoxicity. Dialysis-based procedures for doing these tests in vivo have been described above and microdialysis analysis can be used to verify whether nitric oxide infusion causes Zn²⁺ release into the extracellular fluid in vivo.

### Time Course Experiments in Cerebellar Cortical Brain Slices: Controls

The time course studies should also be performed on cerebellar slices to verify that any release of Zn²⁺ from the brain slices is actually from axon terminals. Unlike the cerebrum, the cerebellum is essentially devoid of zinc-containing axonal boutons, so there can be no synaptic release of Zn²⁺ in the cerebellum. The reason this “negative control” is important is that nitric oxide has an effect on cytosolic zinc-containing proteins in addition to its effect on boutons. Specifically, nitric oxide mobilizes Zn²⁺ from cytosolic proteins in both cerebellum and cerebrum. Thus, on the remote chance that the Zn²⁺ measured in the slice perfusates is “leakage” from the cytosolic pool instead of from bouton release, this is seen in cerebellar slices also.

Time Course Experiments with Lowered Ca²⁺/Mg²⁺ Ratio

Nitric oxide can release transmitter substances by both Ca²⁺ dependent and Ca²⁺ independent mechanisms. Knowing which is operational is important theoretically and for the rational design of drug therapies. This can be determined by examining the time course with lowered Ca²⁺/Mg²⁺ ratio in the bath.

### Magnitude and Time Course of Zinc Release by Peroxynitrite

It is contemplated that the most important moiety in the massive “floods” of Zn²⁺ during excitotoxicty could be the product of superoxide reaction with nitric oxide, peroxynitrite. It is hypothesized that nitric oxide combines with O* to produce peroxynitrite, which is the cause for the release of Zn²⁺. It has been shown that glutamate (Zn²⁺) toxicity is exquisitely sensitive to the adequacy of superoxide scavenging, and that peroxynitrite is even more potent than nitric oxide at releasing Zn²⁺ from thiol ligands. Thus, it is possible that ONOO* is formed when both nitric oxide and O* are elevated after traumatic brain injury plus hypoperfusion. This can be determined by performing the time course experiments using peroxynitrite instead of nitric oxide as the zinc release stimulus.

Microdialysis Analysis of Nitric Oxide-Induced Zinc Release from Hippocampus

The microdialysis analysis can be performed as described above. These experiments will be the first direct in vivo demonstration that Zn²⁺ is released into the extracellular fluid of the brain during any form of excitotoxicity. Verifying that zinc release occurs, and determining when it occurs (with cerebellar control) are important for the rational design of drug therapies.

### Example 3

### Zinc-Mediated Toxicity After Ischemia and Reperfusion

In an in vivo model anesthetized rabbits subjected to ischemia and reperfusion using a neck cuff model, gave a vigorous release of free zinc into brain microdialysates from hippocampal placements at the onset of ischemia and (25 min later) even more dramatically, at the onset of reperfusion. The free zinc in the dialysates went, on average from a baseline of a few nm to a peak release of 50 to 100 nm.

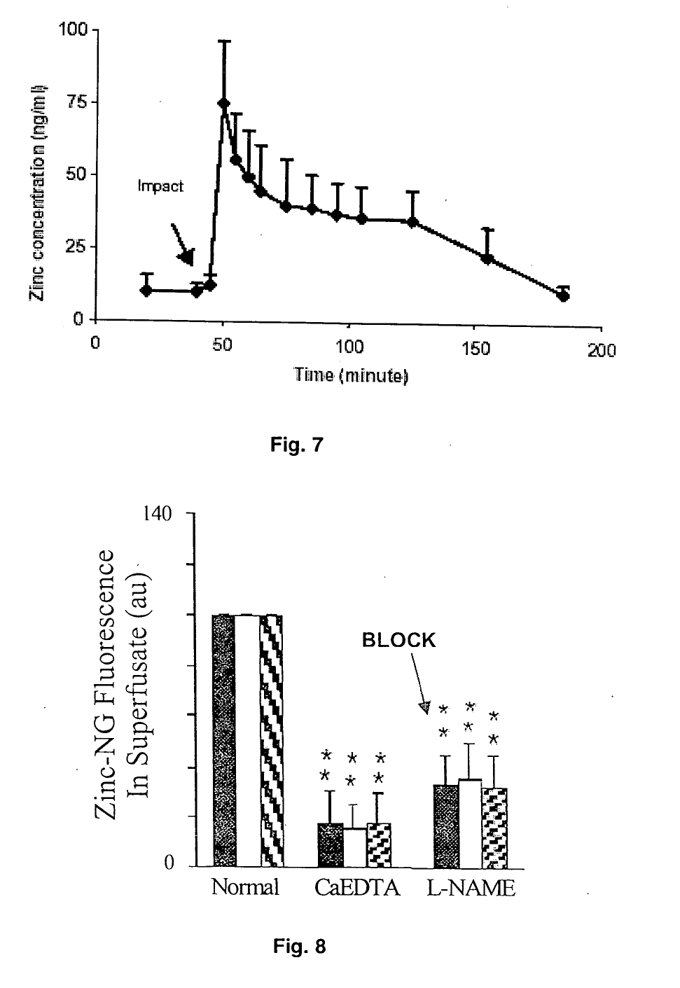

In the in vitro model, the ischemia was translated to the hippocampal slice using the established oxygen-glucose deprivation (OGD) paradigm. Zinc in the extracellular fluids of the slice was monitored using membrane impermeable Newport Green as a sensor for Zn²⁺ in the extracellular fluid as described herein. The release results paralleled the results obtained in vivo from the rabbits. Zinc was released into the extracellular fluid at the onset of OGD, then even more release was observed during reperfusion (FIG. 8).

The entire Zn²⁺ release phenomenon was almost completely blocked when the slices were pretreated with the non-specific NOS inhibitor, L-NAME (1 mM). With the L-NAME treated slices, the amount of Zn²⁺ detectable in the extracellular fluid after OGD and reperfusion was barely higher than in the negative control, in which the Zn²⁺ was chelated by an excess (10 mM) of CaEDTA.

### Zinc-Mediated Toxicity After Head Trauma and Hypoperfusion

To demonstrate the role of nitric oxide in zinc release in a clinically-relevant model of brain damage, a model developed by DeWitt and Prough was utilized which simulates the real world situation of head-injured patients in which initial trauma is often followed by episodes of brain hypoperfusion, with the synergistic trauma and hypoperfusion producing more brain damage than either alone (85). Measurement of zinc translocation actually predicts this synergy in that more neurons showed zinc staining (zinc toxicity) after combined trauma and hypoperfusion than after receiving either injury alone.

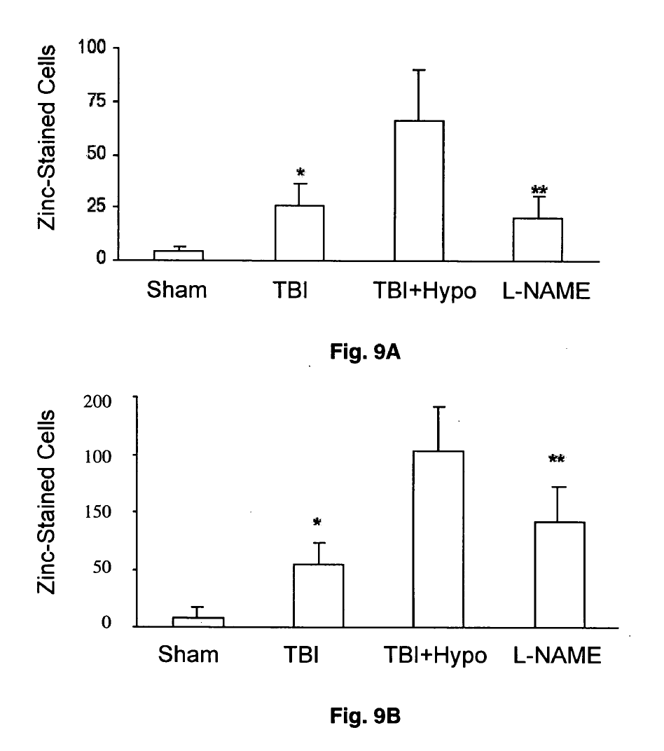

Blocking the synthesis of nitric oxide with the non-specific NOS inhibitor, L-NAME, decreases the release and/or translocation of Zn²⁺ after trauma plus hypoperfusion injury as determined by the number of neurons showing zinc staining (zinc toxicity). As shown in FIGS. 9A-9B, L-NAME caused a substantial reduction in the number of cells stained with zinc. In fact, in both the hilus (FIG. 9A) and the hippocampal cortex (CA3) (FIG. 9B), L-NAME yielded a 50% sparing in the number of zinc-injured (zinc-positive) neurons.

These data strongly support the notion that control of nitric oxide would give clinically important control of zinc-induced neuronal injury. These results can be further confirmed in full, blind, large sample experiments. In addition, inhibitors specific to different nitric oxide synthetases such as iNOS, nNOS, and eNOS can be examined so that the source of the nitric oxide signals pertinent to the Zn²⁺ release can be identified.

### Time Course of Nitric Oxide Release in Trauma-Hypoperfusion Model

If nitric oxide were the trigger for rapid, massive release of Zn²⁺ from the boutons, then one should see nitric oxide signals preceding the Zn²⁺ signals. It is essential to verify that the nitric oxide signals precede or accompany the Zn²⁺ signals in a clinically-meaningful situation, such as the traumatic brain injury-hypoperfusion model. Not only will the lead-lag time, if any, between nitric oxide release and Zn²⁺ release be important theoretically, but it will guide the design of nitric oxide-based therapies. In view of data that indicated traumatic brain injury powerfully sensitized the brain for Zn²⁺ release upon hypoperfusion, these studies will indicate whether that is because the nitric oxide release mechanism has been sensitized or, instead, the Zn²⁺ response to nitric oxide is sensitized. Nitric oxide release can be determined by the nitric oxide electrode method described above.

### Example 4

### Role of NO* and MT in NO*→MT→Zn⁺ Pathway

### Tissue Preparation

### Hippocampal Slice Preparation

Experiments are conducted according to the principles set forth in the Guide for Care and Use of Laboratory Animals (NRC, NIH Publication #74-23). Adult male Sprague dawley rats are anesthetized with ketamine hydrochloride and decapitated. The hippocampi are immersed immediately in ice cold artificial cerebrospinal fluid (ACSF) containing in mM quantities: NaCl, 124; KCl, 1.75; MgSO₄, 1.3; CaCl₂, 2.4; KH₂PO₄, 1.25; NaHCO₃, 26; and dextrose, 10, continuously bubbled with 95% O₂ and 5% CO₂. Transverse hippocampal slices 400 μm thick are prepared using a McIlwain tissue chopper or vibratome. Slices are incubated in a 95% O₂-5% CO₂ saturated interface recording chamber for at least 1 hour before recording at 32° C.

### Dissociated Primary Hippocampal Neuron Culture

Dissociated cell cultures, using hippocampuses instead of cortexes to obtain a higher concentration of gluzinergic neurons, are prepared according to standard methods of Maret (86-91).

### Example 5

### Role of NO* and MT in NO*→MT→.^{o}Zn²⁺ Pathway

### Electrical Stimulation and Recordings

The mossy fiber a CA3 pyramidal neuron responses are induced by stimulating the mossy fiber axons with a 100 μm diameter monopolar Teflon insulated stainless steel wire electrode, exposed only at the tip. Extracellular recordings are obtained using glass micropipettes filled with 2M NaCl, 2-6 MΩ resistance. The recording electrodes are placed at least 500 μm from the stimulating electrodes along the trajectory of the mossy fiber pathway. The recording electrodes are lowered to a distance of 80-100 μm beneath the slice surface. Paired pulse facilitation of the excitatory postsynaptic potential (EPSP) is conducted at 20 and 80 msec interpulse intervals. Slices are accepted for further study when the mossy fiber pathway showed facilitation at the 80 msec interval.

Because of the complex circuitry of area CA3, the metabotropic Glu receptor (mGluR) II agonist 2-(2,3-dicarboxycyclopropyl) glycine (DCG-IV) was used at the end of the experiments to verify that the signal was generated by mossy fiber inputs (92). D-APV (50 μM) was added in ACSF to prevent contamination with the NMDA receptor-dependent pathway converging on CA3 neurons. For inducing mossy fiber via CA3 LTP, test stimuli were delivered to mossy fiber axons every 30 sec (0.03 Hz). The stimulus intensity was set to produce ˜30% of the maximum EPSP. HFS consisted of one train of 100 Hz lasting 2 sec at the intensity that induced the maximum EPSP. The maximal negative initial slope of the mossy fiber via CA3 EPSP was calculated and normalized to 30 min baseline value (defined as 100%).

### Example 6

Role of NO* and MT in NO*→MT→Zn²⁺ Pathway

### Zn²⁺ Imaging

Fluorescence Imaging with Newport Green

For extracellular Zn²⁺ fluorescence imaging hippocampal slices are preloaded with 20 μM Newport Green dipotassium salt (Molecular Probes, Eugene, Oreg.) at room temperature in the dark for at least 30 min. For intracellular Zn²⁺ imaging, the slices were preloaded with 50 μM diacetate ester of Newport Green in 0.5% dimethylsulfoxide containing 0.1% pluronic acid for 1 hr and then washed with ACSF. The Zn²⁺-selective fluorescent dye Newport Green has a Kd of 1 μM for Zn²⁺. Newport Green fluorescence was minimally affected by the presence of Ca²⁺ and Mg²⁺ at physiological concentrations (8, 19). Ca²⁺ or Mg²⁺, up to 10 mM, in the absence of Zn²⁺, had little effect on the dye fluorescence emission.

All experiments were performed at 32° C. under constant ACSF perfusion on the thermostatically heated stage of an inverted microscope (Axiovert 140; Zeiss, Oberkochen, Germany) coupled to a Delta Ram xenon light source (PTI, Manmouth Junction, N.J.) and monochromator set to 506 nM. Emitted light images at 533 nm or greater were acquired at rates of 2-30 Hz through a 10×0.1 numerical aperture objective with an intensified CCD camera (PTI IC-100) and digitized using ImageMaster software (PTI). Autofluorescence was below the detection limits of the camera and photobleaching was negligible under these conditions; neither was subtracted from the data.

To induce the release of Zn²⁺ from mossy fiber terminals, bipolar electrodes 300-500 μm apart are used for electrical stimulation to excite mossy fiber axons. Trains of orthodromic stimuli (100 Hz, 200 μsec pulses at 500 μA unless otherwise noted) of various frequencies are delivered using an S44 stimulator and a PS1U6 photoelectric stimulus isolation unit (Grass Electronics, Quincy, Mass.).

### PAR Zinc Release Assay

For this assay MT is incubated in 20 mM HEPES, pH 7.5 with 100 μM 4-(2-pyridylazo)resorcinol (PAR) and the reaction in the absence and presence of a zinc-releasing agent is followed spectrophotometrically at 500 nm (£(Zn(PAR)₂)=65,000 M⁻¹ cm⁻¹).

### Fluorescence Techniques for Measuring Reactive Species

Hydrogen peroxide is measured with H2DCFDA, 2′,7-dichlorodihydrofluorescein diacetate (Molecular Probes, Eugene, Oreg.). Cells are washed twice with HBSS, resuspended in HBSS, incubated with 5 μM H2DCFDA, and washed three times with HBSS to remove extracellular H2DCFDA. The fluorescence of the oxidized product is then measured at 524 nm (excitation: 504 nm). Nitric oxide also is determined fluorimetrically with the agent diaminofluorescein-2 (93). Superoxide is assayed with a cytochrome c reduction assay (Zhou et al., 2002).

### Example 7

### Role of NO* and MT in NO*→MT→Zn Pathway

### Subcellular Fractions

Preparation of Subcellular Fractions from Mouse Brain

Subcellular fractions are prepared by differential centrifugation and characterized by specific marker enzymes (Graham, 1984). Adenylate kinase is a marker enzyme for the mitochondrial intermembrane space. Cytosolic alcohol dehydrogenase is used to estimate the extent to which the intermembrane space fraction is contaminated with cytosol. Preparations of nuclei are characterized by detection of β-tubulin and the absence of glucose 6-phosphatase activity (Xu et al., 2001). Fractions of the endoplasmic reticulum are assayed for glucose 6-phosphatase. In order to obtain cytosolic fractions that are free from nuclear debris, cells are homogenized with 20 strokes of a Dounce homogenizer in 10 mM Tris-HCl, pH 7.8 and pelleted at 400×g.

### Cell Disruption and Isolation of Nuclei

Cells, >1×10⁷ cells, are harvested and washed three times with phosphate-buffered saline (PBS), collected by centrifugation, suspended in PBS, one volume of lysis buffer (1.28 M sucrose, 40 mM Tris-HCl, pH 7.5, 20 mM MgCl₂, Triton X-100, 4%) and then three volumes of water added all at 4° C. After 10 min on ice, the homogenate is spun at 1300×g for 15 min and then the pellet is resuspended in lysis buffer and spun as above. The pellet contains the purified nuclei. The integrity of nuclei is checked by light microscopy.

Isolation of Mitochondria from Cells

For isolation of mitochondria, cells are disrupted by nitrogen cavitation (Gottlieb and Adachi, 2001). Mitochondria are isolated by differential centrifugation as described below. Samples are processed further for the preparation of microsomes, i.e., smooth and rough endoplasmic reticulum.

### Example 8

### Role of NO* and MT in NO*→MT→Zn²⁺ Pathway

### Intracellular Zinc

### Measurement of Intracellular Zinc

Available cellular zinc is determined fluorimetrically with Zinquin (1-5 μM) (Luminis Pty, Adelaide, South Australia) (94) or Zinpyr-1 (95) in aliquots of cellular fractions that have been used for the determination of MT and T. Owing to its higher quantum yield, Zinpyr-1 can be employed at lower concentration than Zinquin. Concentrations of Zinquin used in earlier publications are likely much too high, e.g., 25 μM (96). Concentrations of 1 μM or lower are used to avoid re-equilibration of zinc between the fluorescent dye and other zinc-binding sites.

These measurements on homogenates are compared with those where the fluorophore has been added to whole cells in order to establish whether or not zinc is released, e.g., from zinc-containing vesicles that occur in some cells, during homogenization of the cells. Fluorescence is calibrated essentially as described by Turan et al. (97) by adding N,N,N′,N′-tetrakis(2-pyridylmethyl)ethylenediamine (TPEN), a membrane-permeable chelating agent, to the cell and by saturating the binding site of the fluorescent dye with extracellular zinc in the presence of the zinc ionophore pyrithione. The zinc fluorophores are non-ratiometric and only relative measurements are possible. This is not a problem since only changes of the intracellular concentration of available zinc are measured.

### Zinc Analysis

Samples are wet-digested with 100 μL nitric acid and 100 μL hydrogen peroxide at 85° C. for 12 h and then analyzed by graphite furnace atomic absorption spectrophotometry or ICP-MS. In general, analytical data are normalized to protein concentration. To avoid bias due to changing protein concentrations, protein concentration is normalized to DNA content.

### Determination of Total Metallothionein (MT+T), T, and MT

The procedure is essentially as described in Yang et al. (98) and employs a very fast derivatization of thiols with ABD-F (7-fluorobenz-2-oxa-1,3-diazole-4-sulfonamide) in the presence of TCEP (tris-2(carboxyethyl)phosphine). The derivatization and analysis are performed on samples with and without added EDTA. The analysis in the presence of EDTA provides total metallothionein (MT+T) while the analysis in its absence provides only thionein T. MT is then calculated as the difference between the two measurements.

The fluorimetric MT/T assay is exquisitely sensitive with a detection limit of femtomolar amounts of the proteins. Therefore, problems in detecting MT/T in extracellular fluid or cells are not anticipated. In order to determine whether or not there is any bias in the measured MT/T ratios when tissue is homogenized, the following controls are performed. Because the recoveries of MT and T added to tissue homogenates are >90% and 70%, respectively (98), samples can be spiked with MT and T during homogenization in order to determine whether or not there is any fast process that would change the ratio.

The derivatization with ABD-F must be performed on fresh tissue as the amount of T increases from 27 to >70% when the sample is stored at −80° C. (98). The reason for this increase may be due to a loss of zinc from MT. Yet, the derivatization with ABD-F is so fast compared with binding of zinc to T that the assay indeed detects T and not MT that could have been formed. Further, in the homogenate MT is less stable than T. The total amount of MT decreases by about 20% after 20 hr at 4° C. (98). Thus, this relatively slow zinc release from MT also does not affect measurements.

Another possibility is that some zinc is released during homogenization, binds to T, and changes the ratio. The fact that T is measured after homogenization indicates that if zinc is indeed released, it is not released in amounts commensurate to affect our results. The reason for the lower recovery of added T compared to MT may be the removal of zinc from some cellular proteins. Nevertheless, whether or not homogenization releases any zinc can be monitored by adding Zinquin (5 μM) in control experiments. It is also critical that dilutions with regard to total protein are kept constant in order not to compare samples where zinc could have dissociated upon dilution. Times at which the assays are performed after breaking up the tissue/cells are also standardized. Furthermore, the MT FRET sensors generate an independent set of analytical data in vivo for comparison.

Expression of Human MT Isoforms in E. coli and Mutagenesis

Using the IMPACT system (intein-mediated purification with an affinity chitin-binding tag, New England Biolabs, Beverly, Mass.), a high-yield E. coli expression system has been developed for the preparation of human MT-2 yielding about 6 mg pure zinc-reconstituted MT per 1 L of culture which is higher than that of any other MT expression system (99). This system has the advantage that T is prepared in situ and then can be used either directly or reconstituted with metals, i.e., T, oxidized T, and MT can all be prepared.

The other human isoforms (MT-1, -3, and -4) also are expressed from a synthetic gene (99). The same system is used to express the two domain peptides of human MT-2, which previously obtained by chemical synthesis and characterized (100) and MTs with site-directed mutations. Mutagenesis is performed by the oligonucleotide-directed dual amber-long and accurate PCR method (Mutant.™.-Super Express Km kit, Takara Shuzo, Kasatsu, Japan) (101).

It has been reported that the redox sensitivity of the alpha-domain of MT-3 results in discrete Zn3- and Zn4-forms (102). Both zinc-containing forms can be prepared by air oxidation. A study of their fluorescence properties may be used to distinguish between the intracellular and extracellular forms of MT-3.

### Preparation and Characterization of Zinc-or Cadmium-MT and T

The concentrations of MTs reconstituted with zinc or cadmium (103) are determined photometrically (ε220=159,000 M⁻¹ cm⁻¹). Sulfhydryl groups are determined spectrophotometrically with 2,2′-dithiodipyridine (ε343=7,600 M⁻¹ cm⁻¹), and zinc content by atomic absorption spectrophotometry. T is prepared from MT and stored in liquid nitrogen (104). T is lyophilized from its frozen solution in 10 mM HCl. When dissolved in buffer, its concentration (ε220=76,000 M⁻¹ cm⁻¹) and sulfhydryl content are determined spectrophotometrically, and it is used immediately for any assay.

### Fluorescent Labeling of MT: Single-Labeled F-MT R-MT and Double-Labeled FRET-MT

Single-labeled at the N-terminus (F-MT) and at the cysteine side chain in the linker region (R-MT) and double-labeled FRET-MT are prepared by established protocol for MT-2 (101). A FRET sensor for the C-terminal alpha-domain was obtained by labeling the same Cys with a fluorophore that serves as a fluorescence acceptor for a tryptophan that has been added to the C-terminus of the MT-2 at C7.

Generally, FRET-MT can be introduced into cells and tissues. In testing with HepG2 hepatomea cells, MT incorporated into the cells co-localizes not with transerin which enters the cell via a clathrin/receptor-mediated endocytosis pathway, but instead with albumin, which most cells take up by caveolae-dependent endocytosis (FIGS. 9A-9D) and use the FRET color changes to track the zincation and de-zincation of the T/MT pair to monitor the action of the T/MT switch at the center of the NO*→MT→Zn²⁺ signaling pathway. The expressed human MT-2 described above has a free N-terminus which is labeled with ALEXA-488 succinimidyl ester. A second fluorescent probe, ALEXA-546 maleimide, is attached at a Cys in the linker region between the domains, i.e., Ser at position 32 is mutated to a Cys.

Finally, a dual-labeled fluorescein/rhodamine-labeled MT-2 was constructed by the same strategy. It exhibits energy transfer between the fluorophores and can be employed as a FRET sensor for monitoring metal binding in the beta-domain, which is the reactive domain in metal transfer experiments. A FRET sensor for the alpha-domain is based on a pair of fluorophores where a C-terminally added tryptophan serves as the donor for the fluorescence acceptor attached at Cys-32. In comparison with a recently designed FRET sensor, which has two green fluorescent protein fluorophores attached at each terminus of the protein and measures zinc release in the cell (105-106), these sensors have the advantages of measuring the metal content precisely and of having a structure that is much closer to that of the native protein, because they bear relatively small fluorophores instead of large proteins at each terminus.

MT-3 fret sensors can be prepared using the same protocol. Fluorescence labeling must be performed on the cadmium-substituted protein, because only the stability of this protein affors specific labeling. All cadmium-containing proteins are converted to the corresponding zinc proteins or apoproteins. The following fluorescently-labeled and unlabeled proteins are made: FRET alpha MT-3: two labels in the alpha domain;

### FRET Beta MT-3: Two Labels in the Beta Domain;

single-labeled MT-3 (rhodamine and fluorescein labels);
single- and double-labeled thionein:
single- and double-labeled domain peptides of MT-3; and
unlabelled MT-3 and T-3.

### Preparation of Mitochondria and Submitochondrial Fractions

Isolation of liver mitochondria is based on the method of Johnson and Lardy (107). In our studies employing this procedure, the P:O (ADP/oxygen consumption) and respiratory control (state 3/state 4 respiration) ratios of different mitochondrial preparations are usually 2.5-2.9 and 6.5-8.5, respectively (108). For localization studies, liver mitochondria are further purified by gradient centrifugation according to Rickwood et al. (109), and for the preparation of submitochondrial fractions the methods of Greenawalt are used (108). Cytosolic contamination of mitochondria, of the intermembrane fraction, and of other submitochondrial fractions is assessed by measuring cytosolic alcohol dehydrogenase activity with ethanol as a substrate (110). Assays for different respiration states in the absence and presence of inhibitors of the respiratory chain and uncouplers have been established using polarographic determination of oxygen (108).

### Example 9

### Role of NO* in Zn²⁺ Signaling During Weak, Strong and Pathological Physiological Activation of Hippocampal Pathways

### Presynaptic Release

For all studies, acutely-prepared hippocampal slices from adult (250+g) rats. The standard preparation is a 400 μM thick section cut on either vibratome or a chopper, collected in iced, Ca²⁺-free medium, then equilibrated at room temperature for 30 min prior to transfer to a temperature-controlled submerged-slice station on an inverted microscope. The slices are maintained in a standard ACSF solution as described in Example 9. To buffer the free zinc and keep it at physiological levels (5 nM ref), a relatively weak and slow-on rate ligand is used, e.g., biccine, which keeps basal zinc rates low, but is too slow to interfere with the zinc signaling transient that occur at synapses (Nicoll et al). For inducing seizures, the procedure of Lea and Sarvey (111) is used.

Primarily between-slice comparisons are used, i.e., applying one drug to one slice and a different drug to the next. One slice cannot be put through the LTP induction or the status epilepticus induction more than once. However, when testing the role of NO* in zinc signaling during weak, slow stimulation, presumably pre, during, and post-drug comparisons may be used on the same slice. For between slice testing, a slice for stimulation of the mossy-fiber axons and recording of the CA3 evoked potential (population spike) is prepared and is instrumented. Threshold and saturation stimulation intensities are determined for the mossy-CA3 population spike.

### Real-Time Method

A fluorescent probe, Newport Green impermeant or ZP4 impermeant for extracellular zinc is bath loaded into the perfusion reservoir and continuously perfused, with recycling, through the chamber. Next, the slice is stimulated at a weak (1.2×threshold), slow (2 per min) rate and fluorescent images are collected and averaged to capture the release of Zn²⁺ into the extracellular fluid. Following the baseline recording, the slice is subjected to an intense “buzz” of stimulation, chosen such that LTP will typically be induced. Fluorescent imaging during the buzz indicates whether detectable amounts of zinc are released. After collecting some post-LTP responses in the fluorescence imaging, the third, pathological intensity of stimulation is imposed to induce seizures.

For the seizure condition, the procedure of Lea and Sarvey (111) is used. Briefly, the slice is stimulated repetitively in the presence of elevated K⁺ until it produces one spontaneous paroxysmal burst. Thereupon, the normal medium, normal K⁺, is restored and subsequent seizure-like bursting is recorded. Fluorescent imaging continues during and after this pathological activation to measure the zinc release and mobilization induced by this activity.

### Before-and-After Method

A “before and after” method may be used to visualize the release of zinc, in which the zinc is stained in situ in the presynaptic vesicles, using TSQ or ZP1, which are both lipophilic, then to measure the intensity of the fluorescence by microfluorimetry. However, in this method the Zn²⁺ probes disrupt internal biochemistry of the vesicle because they all bind too tightly at nM affinity for the millimolar Zn²⁺ concentration of the vesicle. Therefore, it is contemplated that one cannot study zinc release in the presence of the intravesicular probes. Thus, the “before and after” paradigm must be used in which unstimulated slices are compared with previously-stimulated slices, all stained after the stimulation, or non-stimulation, conditions. Although tedious, the method does work (24, 112). As used herein, the before-and-after test determines if the extracellularly released zinc corresponds to a diminution of vesicular zinc.

### Translocation/Mobilization of Free Zinc in Postsynaptic Neurons

The two mechanisms, translocation and mobilization, are linked together empirically because they have the same net effect of raising Zn²⁺ in the cytosol of the postsynaptic neurons. These mechanisms cannot be cleanly separated empirically without the use of ⁶⁵Zn²⁺ which can be followed from extracellular to intracellular compartments. It is contemplated that such ⁶⁵Zn²⁺ tracing is performed to further delineate the movement of postsynaptically-released free zinc.

Monitoring of postsynaptic translocation and mobilization is performed exactly as monitoring presynaptic release, except that zinc mobilization within the postsynaptic neurons is monitored instead of zinc release from terminals. The method requires the sequential measuring of zinc in the at least three separate zinc pools effected in active brain tissue during the synaptic release and the postsynaptic mobilization/translocation of zinc. It is contemplated further that with appropriate zinc probes, which have different excitation or emission wavelengths, that would localize in the different compartments, the presynaptic release and the postsynaptic mobilization may be studied at the same time.

### Change in Levels of Presynaptic Zinc: Pool 1

Pool 1 is by far the most concentrated pool of free zinc but is the most difficult zinc pool to measure without disturbing the physiology of the synapse. This is because of the high concentration of zinc in the vesicles. At an estimated 1-10 mM in the vesicles, any existing fluorescent probe for zinc, with affinities in the low micromolar to low nanomolar, would be 100% saturated in vesicles. In turn, if one put in enough of such a dye to study zinc in the percent occupancy mode, that would completely bind all of the zinc in the vesicles, ruining physiological status of the vesicle. Thus, to study any net loss or gain of vesicular zinc during normal-physiological, strong-physiological, and intense-pathological activation of the tissue, the before-and-after method is used.

Incubate and stabilize A hippocampal slice is incubated and stabilized in the bath. The electrodes are placed on the slice and test responses at a slow, weak intensity are recorded. A stimulation program using normal, strong or pathological intensities continues for 30 minutes or if the slice is a control, nothing is done for 30 minutes.

A stoichiometric excess of a vesicular zinc probe (ZP1 or TSQ) is added to the bath. The intensity of the zinc fluorescence in the neuropil of the tissue is measured microfluorimetrically. Control slices and each of the three types of the stimulated tissues are compared to each other.

### Change in Levels of Synaptically-Released Zinc in the Extracellular Microenvironment of Zinc-Containing Terminals: Pool 2

The “puffs” of free zinc that are released into the extracellular space are relatively easy to measure, using either relatively weak-binding fluorescent probes, e.g., Newport Green at 10 μM KD, or tight-binders, e.g., the apoCA biosensor or the ZNAF probe of Kikuci (113). In the case of weak binders, the increase in signal is presumably due to increased binding of zinc as the zinc concentration rises within the affinity range of the dye; the weak-binding dye shows an “off” signal as the zinc concentration falls. In the case of the tight binders, there is essentially NO* “off” signal, as the amount of bound zinc, i.e., percent occupancy, rises with rising zinc concentration, but will not fall, due to the tight binding, i.e., slow off rate. It is noteworthy that three recent methods using 3 different probes have all estimated the peak level of released zinc to be in the 5-20 μM range.

To study the role of NO* in modulating Pool 2, the synaptically-released zinc, the instrument and slices are stabilized and test stimulation and recording and the establishment of thresholds is as described. The slices are bath loaded with a membrane-impermeable zinc probe, such as Newport Green impermeant. Either a NOS inhibitor (Experimental) or a control compound (control) is added to the bath. Slices are stimulated first at normal-physiological intensity, then at strong-physiological intensity, then at seizure-inducing intensity. During all stimulation epochs, the extracellular zinc is monitored continuously on the confocal microscope for later analysis of the changes in extracellular zinc. Change in Levels of Post-Synaptic “Somatic” Free Zinc in the Cytosol of Dendrites and Spines: Pool 3

Free zinc that shows up in the cytosol of cells is also easy to monitor and quantify due to the fact that there are so-called “trappable” dyes available. These probes have an appendage, typically an ester, attached, which is rapidly cleaved by enzymes in the cytosol, leaving the probe strongly charged and thus “trapped” in the cytosol of the cell. It has been shown empirically that such dyes do not penetrate into the presynaptic vesicles, presumably because they are trapped in the cytosol before they can pass through the vesicular membranes, and that they, therefore, give bright and selective fluorescent reporting of cytosolic zinc. Cytosolic zinc in normal, healthy, resting neurons is normally in the range of 1-4 μM in concentration (114). Therefore, after bath loading of slices, with a “trappable” probe, any zinc signal that arises is readily discriminated from the background. These steps basically follow the steps as described in Pool 2.

### Example 10

### Role of MT-3 in Zn²⁺ Signaling During Weak, Strong and Pathological Physiological Activation of Hippocampal Pathways

### Role of MT-3 in Postsynaptic Zn²⁺ Signaling

Acutely-prepared brain slices are treated exactly as in Example 9, but MT-3 activity are monitored instead of Zn²⁺ signals.

The FRET MT-3 probe is bath-loaded into the slice, using fluorescent imaging of the two wavelengths to track the location of the probe and the ratio between the two peaks to track the degree of zincation. This procedure allows both intracellular or cytosolic and extracellular spaces to be loaded. Subsequent washing, while monitoring fluorescence, is utilized to determine the timing and concentration that yields the maximum intra/extracellular loading of the dye.

Once slices are loaded, the stimulation paradigm described in Example 9, i.e., using first weak, then strong, then pathological stimulation intensities. Preferably, different slices are used for the different stimulation regimens to avoid sequential effects. The endpoint in each stimulation trial is a change in the zincation of the MT-3 in the postsynaptic neurons, as inferred from changes in the fluorescence ratio of the FRET zincation sensor.

For a positive control, an NO* donor, e.g., sperminineNONOate, is introduced to force the dezincation of the MT-3 FRET sensor. For the opposite positive control, Zn²⁺ plus the zinc ionophore pyrithione can be used to fill the cytosol with Zn²⁺. For a negative control, the entire stimulation protocol is performed in the presence of TPEN, which will strip Zn²⁺ off all weak binding sites inside and outside the cells and thus prevent any zincation of MT's during stimulation.

At the highest magnification in confocal microscope, individual giant mossy boutons, individual dendrites and, just barely, giant thorny excrescences on CA3 proximal dendrites can be seen. Higher powered microscopy can be utilized to verify the cellular source(s) of the signals where the presence of FRET-MT-3 changes in the tissue is detected.

### NO* Release of Zn²⁺ From MTs

NOS inhibitors, such as L-NAME and other NOS inhibitors are used to determine the role of NO* in the zincation/dezincation of the FRET MT-3 in hippocampal slices. The methods in Example 9 are used except that MT-3 is monitored instead of Zn²⁺.

### Example 11

### Role of MT-3 in Uptake, Reuptake and Redistribution of Synaptically Released Zinc

### Determining MT Zincation: MT/T Ratio

The degree of MT zincation is measured for the intracellular compartment in dissociated primary hippocampal neurons cultured as described above and in the culture medium representing the extracellular compartment. The MT/T ratio is measured, using the MT fluorometric chemical modification method described herein for the culture medium prior to incubating tissue in it, for the conditioned medium and for the intracellular fluids of the cultured hippocampal cells. Next, the total concentration of MT, both extracellular and intracellular fluid compartments, is measured as described in Example 8.

### Determination of Transmembrane Movement of MT-3

Hippocampuses are removed from young (P2) rat pups. The cells are dissociated, plated out and allowed to grow until a sufficient culture is obtained. FRET MT is added to different flasks containing cells+medium for 10, 100 and 1000 minutes. The medium is removed from each flask, the cells are harvested and the cytosolic fraction from the cells also is harvested. The abundance of MT is measured by fluorimetry and the zincation or FRET ratio of the MT in both fractions at each time point are both measured in the extracellular and intracellular fractions at each time point as previously described. These results may be used in repeated assays to narrow the time window to determine the critical time interval during which most of the MT-3 enters the cells.

Variation of temperature, such as lowering 4° C. degrees or more and monitoring the transmembrane movement may indicate if the movement is active or passive. Additionally, it is possible to determine where in the endocytotic pathway metals are released and what the destiny of Zn-MT is in this pathway by blocking each step in the pathway with specific inhibitors. Ouabain (1 mM) inhibits the acidificaton of early endosomes and bafilomycin (100 nM) blocks the pathway leading to lysosomes.

Furthermore, it is possible to determine into which particular type of cell or cellular region the MT moves. The flask cultures are supplemented by parallel cultures grown in couture chambers with cover-slip bottoms for viewing in the confocal microscope. Once the time course and energy dependence, if any, of the movement into cells are established, viewing in the confocal microscope will show into which regions or organelles of which cell-types the MT-3 has gone. Moreover, as the confocal microscope has 3 emission channels, it can be assessed quantitatively whether the FRET MT-3 reaches different degrees of zincation in different cellular compartments, i.e., nuclear localization, localization in the Golgi apparatus and localization in generic endosomes, as well as localization in, for example, perikaryon, dentrites or, just barely, axonal boutons. Double immunostaining would be required to visualize greater detail.

### Movement of MT Out of Cells

It is contemplated that neurons may secrete MT. This might occur constitutively, via regulated secretion or even at synaptic terminals. To examine MT release, the medium for the cultured hippocampal cells is changed to fresh, synthetic medium. High potassium (40 mM) is added to depolarize cells in some flasks while other flasks not receiving high potassium are controls. The medium from each flask is collected after a predetermined period and the total MT is measured to examine for effects of high potassium.

If high potassium increases efflux of MT into the medium, release via exocytosis may be tested. Negative controls are tested in Ca²⁺ free medium, tested in cold and tested in lamotrigine medium. Positive controls are tested in with alpha latrotoxin and with glutamate. It also can be determined if the release is thionein or metallothionein by measuring the T/MT ratio of the effluxing protein.

The following references are cited herein:
1. Martinez-Guijarro et al., 1991. J Neurocytol 20:834-43.
2. Frederickson C J, Bush A I. 2001. Biometals 14:353-66.
3. Casanovas-Aguilar et al., 1998: J Chem Neuroanat 15:97-109.
4. Sorensen et al., 1995. Exp Brain Res 105:370-82.
5. Frederickson et al., 1994. Biol Signals 3:127-39.
6. Asao S Y, Chung S H. 1984. Nature 308:734-6.
7. Howell et al., 1984. Nature 308:736-8.
8. Li et al., 2001. J Neurophysiol 86:2597-604.
9. Frederickson et al., 2003. Zn2+ and glutamate signaling in the ischemic brain.
10. Xie X M, Smart T G. 1991. Nature 349:521-4.
11. Mollereau et al., 2001. Nature 412:911-3.
12. Erben et al., 2002. Mol Endocrinol 16:1524-37.
13. Cole et al., 2000. Epilepsy Res 39:153-69.
14. Mitchell C L, Barnes M I. 1993. Neurotoxicol Teratol 15:165-71.
15. Mitchell et al., 1990. Brain Res 506:327-30.
16. Dominguez et al., 2003. Neuroscience 116:791-806.
17. Land P W, Akhtar N D. 1999. Somatosens Mot Res 16:139-50.
18. Brown C E, Dyck R H. 2002. J Neurosci 22:2617-25.
19. Li et al., 2001. J Neurosci 21:8015-25.
20. Lu et al., 2000. Synapse 38:187-97.
21. Itoh M, Ebadi M. 1982. Neurochem Res 7:1287-98.
22. Klitenick et al., 1983. Anal Chem 55:921-3.
23. Yokoyama M, Koh J, Choi D W. 1986. Neurosci Lett 71:351-5.
24. Frederickson et al., 1988. Brain Res 446:383-6.
25. Sloviter R S. 1985. Brain Res 330:150-3.
26. Haug F M et al., 1971. J Comp Neurol 142:23-31.
27. Frederickson et al., 1989. Brain Res 480:317-21.
28. Frederickson C J. 1989. Int Rev Neurobiol 31:145-238.
29. Sorensen et al., 1998. Brain Res 812:265-9.
30. Tonder et al., 1990. Neurosci Lett 109:247-52.
31. Suh et al., 2000. Brain Res 852:268-73.
32. Suh et al., 2001. Neuroreport 12:1523-5.
33. Yin et al., 2002. J Neurosci 22:1273-9.
34. Choi et al., 1998. Annu Rev Neurosci 21:347-75.
35. Cuajungco M P, Lees G J. 1997b. Neurobiol Dis 4:137-69.
36. Weiss et al., 2000. Trends Pharmacol Sci 21:395-401.
37. Jiang et al., 2001. J Biol Chem 276:47524-9.
38. Sensi S L, Yin H Z, Weiss J H.2000. Eur J Neurosci 12:3813-8. Volume 10, Number 1, 2004.
39. Koh J Y. 2001. Mol Neurobiol 24:99-106.
40. Lees G J, Cuajungco M P, Leong W. 1998. Brain Res 799:108-17.
41. Cuajungco M P, Lees G J. 1998a. Brain Res 799:97-107.
42. Koh et al., 1996. Science 272:1013-6.
43. Jhamandas et al., 1998. Amino Acids 14:257-61.
44. Chemy et al., 2001 Neuron 30:665-76.
45. Cuajungco M P, Lees G J. 1996 Neuroreport 7:1301-4.
46. Santore et al., 2002 Toxicol Pharmacol 133:271-85.
47. Lee et al., 2002a Neuroscience 115:871-8.
48. Hellmich et al., 2003. Neuroscience. Forthcoming.
49. Lee J Y, Palmiter R D, Koh J Y. 2002 Soc Neurosci Abs.
50. Cuajungco, M P, Lees G J. 1997a. Brain Res Rev 23:219-36.
51. Cherny et al., 1999. J Biol Chem 274:23223-8.
52. Regland et al., 2001 Dement Geriatr Cogn Disord 12:408-14.
53. Lee et al., 2002b Proc Natl Acad Sci USA 99:7705-10.
54. Lee et al., 2000. J Neurosci 20:RC79.
55. Frederickson et al., 2002 Neuroscience 115:471-4.
56. Frederickson et al., 2002 J Histochem Cytochem 50:1659-62.
57. Perez-Clausell J, Danscher G. 1985 Brain Res 337:91-8.
58. Cuajungco M P, Lees G J. 1998b Brain Res 799:118-29.
59. Togashi et al., 1998 Neurosci Lett 240:53-7.
60. Yin H Z, Weiss J H.1995 Neuroreport 6:2553-6.
61. Sheline et al., 2002. Neurobiol Dis 10:41-53.
62. Persechini et al., 1995. Biochemistry 34(46):15091-5.
63. Kim Y H, Koh J Y. 2002. Exp Neurol 177(2):407-18.
64. Maret W. 1995 Neurochem Int 27:111-7.
65. Bogumil et al., 1996 Eur J Biochem 238:698-705.
66. Chen et al., 2002 Biochemistry 41:8360-7.
67. Hidalgo et al., 2001 Brain Res Bull 55(2):133-45.
68. Sensi et al., 1997 J Neurosci 17:9554-64.
69. Tsuda et al., 1997 J Neurosci 17:6678-84.
70. Colvin et al., 2000 J Nutr 130:1484 S-7S.
71. McDonald et al., J Physiol. Pharmacol., 53:555-69 (2002). [0304] 72. Li et al., Psychopharmacology, Feb. 13, 2003 e-publication.
73. Limbourg et al., J Clin. Invest., 110:1729-38 (2002).
74. Mitral et al., Am. J Physiol. Heart Circ. Physiol., 279:H2649-57 (2000).
75. Alderton, et al., 2001 Biochem J 357:593-615.
76. Aghajanian and Rasmussen, Synapse, 3:331-8 (1989).
77. Richerson and Messer, Exp Neurol, 131:133-43 (1995).
78. Thompson et al., J Neurosci Meth. 96:35-45 (2000).
79. Conroy et al, Anesthesiology 90:844-54 (1999).
80. Frederickson et al, J Neurosci. Meth. 20:91-103 (1987).
81. Frederickson et al, J Chem. Neuroanat. 5:521-30 (1992).
82. DeWitt et al., J Neurotrauma. 14:219-229 (1997).
83. Howell et al., Nature 308:736-738 (1984).
84. Suh et al, Brain Research 879:7-12 (2000).
85. Dewitt and Prough, Crit Care Med. 28:1-2 (2000).
86. Maret W. 1994. Proc. Natl. Acad. Sci. U.S.A 91:237-241.
87. Maret W. 1995. Neurochem. Int. 27:111-117.
88. Maret W. 2003. J. Nutr. 133:1460 S-1462S.
89. Maret et al., 1999a Proc. Natl. Acad. Sci. U.S.A 96:1936-1940.
90. Maret, et al., 1997 Proc. Natl. Acad. Sci. U.S.A 94:2233-2237.
91. Maret et al., 2001 Chem. Biol. Interact. 130-132:891-901.
92. Yoshino et al., 1996 Neurosci Lett. 207(1):70-72.
93. Kojima et al. 1998 Anal Chem. 70(13):2446-2453.
94. Zalewski et al., 1993 Biochem J. 296 (part2):403-408.
95. Walkup et al., 2000 J Am Chem Soc. 122:5644-5645.
96. Ho et al., 2000 Biochem Biophys Res Commun. 268(1):148-54.
97. Turan et al., 1997. Am J Physiol. 272(5 Part 2):H2095-2106.
98. Yang et al., 2001 Proc. Natl. Acad. Sci. U.S.A. 98:5556-5559.
99. Hong et al., 2001Protein Expr. Purif. 21:243-250.
100. Jiang et al., 2000 Proc. Natl. Acad. Sci. U.S.A. 97:2503-2508.
101. Hong et al., 2003 Proc. Natl. Acad. Sci. U.S.A 100:2255-2260.
102. Roschitzki B and Vasak M. 2003 Biochemistry 42(32):9822-8.
103. Vasak, M. 1991. Methods Enzymol. 205:41-44.
104. Jacob et al., 1998. Proc. Natl. Acad. Sci. U.S.A 95:3489-3494.
105. Pearce et al., 2000b J. Nutr. 130:1467 S-1470S.
106. St Croix et al., 2002 Am. J. Physiol Lung Cell Mol. Physiol 282:L185-L192.
107. Walter et al., 1967 J Biol Chem 242(21):5014-8.
108. Ye et al., 2001. Proc. Natl. Acad. Sci. U.S.A 98:2317-2322.
109. Rickwood et al., 1982 Anal Biochem 123(1):23-31.
110. Wagner et al., 1983 Biochemistry 22(8):1857-63.
111. Lea P M and Sarvey J M. 2003. Epilepsy Res. 53:207-215.
112. Varea et al., 2001. J. Neurosci. Methods 110:57-63.
113. Ueno et al., 2002 J. Cell Biol. 158:215-220.
114. Outten C E and O'Halloran T V. 2001. Science 292:2488-2492.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for treating a subject with a clinical condition associated with zinc-mediated neurotoxic injury, said method comprising administering to the subject in need of such a treatment a therapeutically effective amount of a compound that reduces the amount of zinc release from the neurons.

2. The method of claim 1, wherein the compound reduces the amount of zinc release by inhibiting nitric oxide synthesis.

3. The method of claim 1, wherein the compound reduces the amount of zinc release by reducing the level of nitric oxide that induces release of zinc.

4. The method of claim 1, wherein the clinical condition comprises a zinc-mediated brain injury.

5. The method of claim 4, wherein the zinc-mediated brain injury comprises stroke, head trauma, ischemia, or seizure.

6. The method of claim 1, wherein the compound comprises 7-nitroindazole, S-methyl-1-thiocitrulline, the protein PIN, 2-amino-5,6-methyl-4H-1,3-thiazine, N⁶-iminoethyl-L-lysine, N⁶-iminoethyl-L-lysine, N-(3-aminomethyl)benzyl acetamidine, or S—[2-amino-(1-iminoethylamino)-5-thioheptanoic acid]

7. A method for preventing a clinical condition associated with zinc-mediated neurotoxic injury, said method comprising administering to an individual susceptible to trauma-induced excitotoxicity a compound that reduces the amount of zinc release from neuronal cells in response to the trauma-induced excitotoxicity, thereby preventing the clinical condition associated with zinc-mediated neurotoxic injury.

8. The method of claim 7, wherein the compound reduces the amount of zinc release by inhibiting nitric oxide synthesis such that reduction of nitric oxide synthesis reduces release of zinc from neuronal cells thereby preventing the clinical condition associated with zinc-mediated neurotoxic injury.

9. The method of claim 8, wherein the compound inhibits nitric oxide synthetic activity of (i) neuronal nitric oxide synthase, (ii) inducible nitric oxide synthase, (iii) or a combination thereof.

10. The method of claim 7, wherein the compound comprises 7-nitroindazole, S-methyl-1-thiocitrulline, the protein PIN, 2-amino-5,6-methyl-4H-1,3-thiazine, N⁶-iminoethyl-L-lysine, N⁶-iminoethyl-L-lysine, N-(3-aminomethyl)benzyl acetamidine, or S—[2-amino-(1-iminoethylamino)-5-thioheptanoic acid].

11. The method of claim 7 further comprising administering a second compound to improve cerebral blood flow.

12. The method of claim 11, wherein the second compound increases the activity of endothelial nitric oxide synthase.

13. The method of claim 12, wherein the second compound comprises simvastatin, 170-estradiol, a corticosteroid, endothelin, or AT2 receptor agonists.
